(19) United States
(12) Patent Application Publication
(10) Pub. No.: US 2003/0044946 A1
(43) Pub. Date: Mar. 6, 2003

(54) Genes, mutations, and drugs that increase cellular resistance to damage and extend longevity in organisms from yeast to humans

(76) Inventor: Valter D. Longo, Los Angeles, CA (US)

Correspondence Address: Rajiv, Yadav, Ph.D., Esq., McCutchen, Doyle, Brown & Enersen, LLP, Three Embarcadero Center, 18th Floor, San Francisco, CA, 94111, US

(21) Appl. No.: 10/116,722

(22) Filed: Apr. 3, 2002

Related U.S. Application Data
(60) Provisional application No. 60/281,213, filed on Apr. 3, 2001.

(51) Int. Cl. C12N 9/99; C07H 21/04; A61K 48/00

(52) U.S. Cl. 435/184; 514/44; 536/23.2

(57) ABSTRACT

In accordance with the present invention there is disclosed a complete molecular pathway that regulates aging and longevity in yeast and evidence for the conservation of this pathway and mechanisms in organisms ranging from yeast to humans. This invention also identifies novel molecular mechanisms of aging in eukaryotes and provides new compositions and methods for the development of drugs that prevent and treat diseases and disorders associated with aging and extend the life-span of humans.

### RELATED APPLICATION DATA

This application claims priority to U.S. provisional patent application No. 60/281,213 filed Apr. 3, 2001.

### RIGHTS OF FEDERAL GOVERNMENT

This work was supported by the National Institutes of Health Grant DK46828 and AG 08761-10. The United States Government may have certain rights in this invention.

### FIELD OF THE INVENTION

This invention relates to methods and compositions for extending the longevity of and preventing/treating aging-dependent diseases in eukaryotes.

### BACKGROUND OF THE INVENTION

It goes without saying that combating aging is a cherished goal of human endeavor. Resisting aging may allow life-span extension. Furthermore, numerous diseases and disorders are associated with aging. Diseases which show age-dependent onset of symptoms include Alzheimer's disease, Pick's disease, Huntington's disease, Parkinson's disease, adult onset myotonic dystrophy, multiple sclerosis, adult onset leukodystrophy, diabetes mellitus, arteriosclerosis, and cancer.

Thus, postponing aging may prevent many diseases and disorders and, therefore, compositions and methods for extending life-span or fighting the consequences of aging have great utility. The identification of the molecular pathways that regulate aging and age-related diseases in humans is very complex. By contrast, the simple eukaryote yeast Saccharomyces cerevisiae is very well studied at the molecular and genetics level.

### SUMMARY OF THE INVENTION

Methods are provided for the identification of the genes and drugs that increase the resistance of human cells to aging and insults, such as oxidative stress and DNA mutations, which lead to therapies that delay or prevent age-related diseases including cancer, Alzheimer's Disease, and Parkinson's Disease.

This invention describes a complete molecular pathway that regulates aging and longevity in yeast and provide evidence for the conservation of this pathway and mechanisms in organisms ranging from yeast to humans. This invention also identifies novel molecular mechanisms of aging in eukaryotes and provides new compositions and methods for the development of drugs that prevent and treat diseases and disorders associated with aging and extend the life-span of humans.

In one aspect the invention provides an agent for extending the life-span of a eukaryote, wherein said agent modulates a pathway which involves the participation of a product of at least one gene selected from the group consisting of a ras gene, adenylate cyclase, SOD2, Sch9, MSN2, MSN4, RIM15 and homologs thereof. In this and other aspects of the invention, the eukaryote can be higher eukaryote such as a mammal, e.g., a human being. In another aspect the invention provides a n agent for extending the life-span of a eukaryote, wherein said agent modulates a signal transduction pathway that regulates multiple stress resistance systems. In yet another aspect the invention provides a n agent for extending the life-span of a eukaryote, wherein said agent modulates a signal transduction pathway that regulates SOD2 activity. The invention also provides a n agent for extending the life-span of a eukaryote, wherein said agent regulates the expression of genes encoding for heat shock proteins, genes encoding for superoxide dismutases, catalase, or DNA repair genes (DDR2). The invention further provides an agent for extending the life-span of a eukaryote, wherein said agent modulates a pathway that depends on the activity of at least one polypeptide selected from the group consisting of Msn2, Msn4, Rim-15 and homologs thereof. The invention additionally provides a n agent for extending the life-span of a eukaryote, wherein said agent modulates a pathway that is activated in response to glucose or other nutrients.

In another aspect the invention provides a method for increasing the life-span of a eukaryote, the method comprising contacting the cell of the eukaryote with the afore-mentioned agents.

The invention provides, in another aspect, system for studying the aging and death of a eukaryote, the system comprising a long-lived yeast mutant.

The invention also provides a method for extending the life-span of a eukaryote, the method comprising modulating a pathway which involves the participation of a product of at least one gene selected from the group consisting of a ras gene, adenylate cyclase, SOD2, Sch9, MSN2, MSN4, RIM15 and homologs thereof. In another aspect, the invention provides a method for extending the life-span of a eukaryote, the method comprising modulating a signal transduction pathway that regulates multiple stress resistance systems. In yet another aspect, the invention provides a method for extending the life-span of a eukaryote, the method comprising modulating a signal transduction pathway that regulates SOD2 activity and superoxide damage in the mitochondria. The invention further provides a method for extending the life-span of a eukaryote, the method comprising regulating the expression of genes encoding for heat shock proteins, genes encoding for catalase, superoxide dismutases, and genes involved in DNA repair. The invention additionally provides a method for extending the life-span of a eukaryote, the method comprising modulating a pathway that depends on the activity of at least one polypeptide selected from the group consisting of Msn2, Msn4, Rim-15 and homologs thereof. The invention further provides a method for extending the life-span of a eukaryote, the method comprising modulating a pathway that is activated in response to a nutrient and is down-regulated during periods of starvation. In one aspect the nutrient is glucose.

### BRIEF DESCRIPTION OF THE DRAWINGS

FIG. 1 is a plot of Survival of yeast cells v. time in days. FIG. 1A is a plot survival of wild type (DBY746), and transposon mutagenized cyr1::mtn (Tn3-24), and sch9::mTn (Tn3-5) yeast cells. FIG. 1B is a plot of survival of wild type and sch9Δ. FIG. 1C is a plot of survival of sch9Δtransformed with wild type SCH9 or with a mutated sch9 encoding for a catalytically inactive proteins (Sch9_{K441A}, Sch9_{D556R}).

FIG. 2A is a plot of survival of wild type and cyr1::mTn mutants lacking either the stress-resistance genes MSN21MSN4 or RIM15. FIG. 2B is a plot of survival of wild type and sch9Δ mutants lacking either MSN2/MSN4 or RIM15.

FIG. 3A shows two photographs of yeast cells removed from day 1 post-diauxic phase cultures spotted onto YPD plates and incubated at 30° C. (control) or 55° C. (heat-shocked) for one hour. Pictures were taken after a 4-day incubation at 30° C. FIG. 3B shows the survival of cells incubated with hydrogen peroxide (100 mM) for 30 minutes. FIG. 3C shows the survival yeast cells treated with 20 μM of the superoxide/H₂O₂-generating agent menadione for 60 minutes.

FIG. 4A shows the mitochondrial aconitase percent reactivation after treatment of whole cell extracts of yeast removed from day 5-7 cultures with agents (iron and Na₂S). FIG. 4B shows the death rate reported as the fraction of cells that lose viability in the 24-hour period following the indicated day.

FIG. 5 shows the Yeast Sch9 serine/threonine kinase putative catalytic domain aligned with other proteins.

Yeast Sch9 serine/threonine kinase putative catalytic domain was aligned with C. elegans AKT-1a (GenBank accession number MC62466)/AKT-2 (GenBank accession number AAC62468), Drosophila AKT-1 (GenBank accession number MF55276), mouse AKT (GenBank accession number S33364)/AKT-2 (GenBank accession number Q60823) human AKT-1 (GenBank accession number A39360)/AKT-2 (GenBank accession number A46288) using DNAssist. Identical and similar residues are shaded in red and green, respectively. Dashes indicate gaps introduced to align the sequences. The Sch9 kinase domain is 47-50% identical to those of all the proteins analyzed.

FIG. 6 shows the results of the experiments of Example 5. It shows that mitochondrial SOD (SOD2) is required for the chronological lifespan extension of sch9Δ (PF102) and cyr1::mtn (PF101) mutants. Cells were grown to saturation (reaching a density of approximately 1×10⁹ cells/flask) in minimal SDC medium and were allowed to incubate in the same medium after reaching the post-diauxic phase. FIG. 6A shows survival of the wild type (DBY746), sod2Δ (EG110) sch9Δ (PF102) and sch9Δ lacking SOD2 (PF106). FIG. 6B shows survival of wild type and cyr1::mtn (PF101) and cyr1::mTn lacking SOD2 (PF105) (p<0.05 for cyr1::mtn sod2Δ vs wild type or cyr1::mTn, Two-Factor ANOVA). The average of two independent experiments with duplicate samples is shown for FIGS. A and B. FIG. 6C shows Northern blot of RNA prepared from exponentially growing, day 5 post-diauxic phase, and day 6 post-diauxic phase cultures of wild type, cyr1::mTn and sch9Δ mutants probed for SOD2. Compared to wild type controls, SOD2 expression in sch9Δ mutants was 3.5 and 8 fold higher at days five and six, respectively. Equal RNA loading was confirmed by ethidium bromide staining after electrophoresis (bottom panel). The experiment was performed twice with similar results.

FIG. 7 shows the results of the experiments of Example 6. It shows the results of the study of life span of SOD overexpressors. Yeast strain DBY746 transformed with the indicated multicopy plasmids (YEp351 and YEp352) either vector-only or carrying cytosolic CuZnSOD (SOD1), mitochondrial MnSOD (SOD2) or cytosolic catalase (CTT1) were tested for survival as described, FIG. 7A shows survival of DBY746 SOD1CTT1 and SOD1SOD2 double overexpressors, FIG. 7B shows survival of DBY746 CTT1 and SOD2 single overexpressors, FIG. 7C shows survival of DBY746 SOD1 overexpressors. Each overexpressor is shown in the same figure as its specific plasmid control (SOD1=YEp352, SOD2 and CTT1=YEp351), FIG. 7D shows the results of an experiment in which strain DBY746 was incubated in the presence of the respiratory inhibitors that reduce mitochondrial superoxide generation FCCP (4 μM) or NaCN (0.25 mM). Viability was measured at the indicated times (at day 9, p<0.05 Students t-test, 5 experiments). To avoid the selection of strains with mutations that increase or decrease survival independently of SODs during the transformation, the experiments with each DBY746 overexpressor strain were performed between 6 and 10 times using transformants obtained from 3 separate transformations, all of which behaved similarly. For each graph, all experiments were averaged; bars show the standard error for each time point. Experiments with SOD1SOD2 and SOD1CTT1 overexpressors in the SP1 parent strain were performed twice with double samples grown independently. The p value calculated by Two-Factor ANOVA using all the viability points and comparing to the appropriate vector control was <0.05 for all the overexpressors.

FIG. 8 shows the results of the experiments of Example 7. It shows the results of the study of aconitase activity in wild type (HM) and SOD1SOD2 overexpressors (LM). Extracts from 5 independent wild type cultures with high mortality (HM) and 5 SOD1SOD2 cultures with low mortality (LM) at day five (2 studies) were assayed for aconitase activity. FIG. 8A shows the percent survival from day 3 to day 9 for the LM and HM groups is shown in the left panel and mortality at day 3-7 is shown in the right panel (p<0.05). For the LM group, mortality at day 5 ranged from 0 to 0.37 (av.=0.17±0.076). For the HM group mortality at day 5 ranged from 0.44 to 0.9 (av.=0.77±0.085). Values are mean±SEM. FIG. 8B shows the aconitase activity in the LM and HM groups expressed as micromoles of cis-aconitate consumed/min/mg (left panel) and aconitase fold increase in activity in the presence of the reactivation agents Fe³⁺ and Na₂S (right panel) (p<0.05 between HM and LM at day 5). Values are mean±s.e. FIG. 8C shows percent survival of wild type cells, coq3Δ mutants (unable to synthesize coenzyme Q of mitochondrial complex III and therefore unable to respire) and of wild type cells treated with agents that increase the generation of mitochondrial superoxide (1 μM antimycin A and 1 mM paraquat. Treatment with antimycin A or paraquat causes the inactivation of aconitase (data not shown).

FIG. 9 shows the results of the experiments of Example 8. It shows the results of the study of survival of wild type and ras2Δ mutants in the post-diauxic phase. The percent survival is shown for: FIG. 9A—Wild type (SP1, closed symbols) and ras2Δ (KP1, open symbols) yeast populations. Experiments were performed 3 times with similar results. A representative experiment with the average of duplicate wild type and ras2Δ populations is shown. The survival for the ras2 strain was significantly longer than that of wild type as determined by ANOVA analysis (p<0.05). FIG. 9B shows the survival of wild type (DBY746) and ras2Δ (EG 252) in the post-diauxic phase. The percent survival is shown. A representative experiment with the average of four populations for each strain is shown. The experiment was repeated 3 times with similar results. Similar results were also seen with another ras2 isolate made in the same background. The survival for each of the ras2Δ isolates was significantly longer than that of wild type as determined by ANOVA analysis (p<0.05). FIG. 9C shows the survival data for wild type (SP1) and RAS2val19 mutants with constitutive active Ras2 (TK1611 R2V). A representative experiment is shown. The experiment was repeated twice with similar results.

FIG. 10 shows the results of the experiments of Example 9. It shows the superoxide toxicity and survival of ras2Δ mutants. FIG. 10A: Wild type (DBY746) and ras2Δ (EG252) cells were grown in SDC to which 1 mM paraquat (superoxide-generating agent) was added after 24 hours. Viability was measured at days 5 and 7. A representative experiment with triplicate samples is shown. The experiments was repeated 3 times with similar results. FIG. 10B: Chronological life span for wild type cells (DBY746) and mutants lacking either RAS2 (EG252) or transcription factors Msn2/4 (PF103) or both (PF107). The experiment was performed three times in duplicate. The average of six samples is shown (p<0.05 for ras2Δmsn2/4Δ compared to ras2Δ). FIG. 10C: Chronological life span for wild type cells and strains lacking either mitochondrial SOD (EG110), Ras2 (EG252), or both (PF104). The experiment was performed twice. The average of 8 independent samples is shown (p<0.05 for ras2Δ sod2Δ compared to ras2Δ or wt).

FIG. 11 shows the results of the experiments of Example 10. It shows the metabolic rates for the long-lived mutants: FIG. 11A: Oxygen consumption for wild type strain DBY746 and ras□□, cyr1::mTn, and sch□□ mutants generated in the DBY746 background (EG252, PF101, PF102). A representative experiment with the average of three independent samples for each strain is shown. FIG. 11B: Oxygen consumption for strain SP1 and ras2□ mutants generated in the SP1 background (KP-1b). A representative experiment with the average of three independent samples for each strain is shown. Cells were inoculated at an initial OD₆₀₀ of 0.2 and aliquots were removed and tested at the indicated times. The point at which the cells reach an OD₆₀₀ of 1 was taken as Day 0.

FIG. 12 shows the results of the experiments of Example 11. It shows the loss of mitochondrial function in wild type yeast. A DBY746 isolate chosen because of its particularly high mortality rate was grown in SDC medium and switched to water on day three. Incubation in water prolongs survival and allows the long-term monitoring of the IRC (colonies formed in carbon sources that require respiration as percent of viable cells). At the times indicated aliquots were plated onto YPD (glucose) and YPG (glycerol) plates. FIG. 12A: Viability on YPD plates, FIG. 12B: average IRC from 3 experiments and 6 independent cultures. Values are means±SEM. The results of two tailed student t-tests of the IRC for each data points between days 5 and 18 against the IRC at day 3 gave p<0.05.

FIG. 13 shows the results of the experiments of Example 12. It shows the time-dependent release of proteins into the medium by wild type and long-lived yeast. FIG. 13A: Concentration of proteins released into the medium by wild type controls (DBY746 351-352) and SOD1SOD2 overexpressors. FIG. 13B: Age-dependent loss of CFU (% decrease) vs. the concentration of proteins released into the medium by dead and damaged wild type DBY746-351-352 cells.

FIG. 14 shows a model for the regulation of stress-resistance and aging in yeast. Glucose activates the Cyr1/cAMP/PKA pathway, in part, via the G-protein coupled receptor Gpr1 and activates Sch9 by an unknown mechanism. Cyr1/cAMP/PKA inactivates stress-resistance transcription factors Msn2/Msn4, which regulate the expression of many stress resistance genes including heat shock proteins, catalase, and MnSOD. Activation of Sch9 results in a major decrease in stress-resistance either via Rim15 and/or Hsp99 or via and unidentified effector. Mutations that decrease the activity of Ras2,(ras2Δ), Sch9 (sch9::mTn and sch9Δ) and Cyr1 (cyr1::mTn) extend the chronological life span by activating stress resistance proteins Msn2, Msn4, and Rim15, decreasing the levels of mitochondrial superoxide, delaying aconitase inactivation and by other unknown mechanisms.

FIG. 15 provides in chart form the proposed mechanisms of aging for several organisms based on the results of this work.

### DETAILED DESCRIPTION OF THE PREFERRED EMBODIMENTS

In accordance with the invention, based on the remarkable phenotypic similarities between yeast ras2 mutants and long-lived nematodes, fruit flies, and mice with mutations in signal transduction proteins, the fundamental mechanism of aging is conserved from yeast to man and, therefore, long-lived yeast mutants are used to elucidate mechanisms that extend longevity in higher eukaryotes.

Yeast provides an ideal organism to study aging It has a life-span of days and has a genome size less than 1/100^{th} of a mammal. In accordance with the invention, the mechanisms and pathways that describe chronological aging (defined below) in yeast provide compositions and methods for extending the life span of eukaryotes, including higher eukaryotes such as mammals.

As a first step in using yeast to study aging, the two types of aging exhibited by yeast must be recognized. Yeast exhibits two types of aging: replicative aging and chronological aging. The first type of aging is observed when microorganisms encounter an ample source of nutrients, in which case they typically divide rapidly, reach a state of overcrowding and then spend the vast majority of their life cycle in stationary phase (Werner-Washburne, M., Braun, E., Johnston, G. C. & Singer, R. A. (1993) Microbiol. Rev. 57, 383-401; Zambrano, M. M. & Kolter, R. (1996) Cell 86, 181-4). Yeast incubated in rich glucose medium (YPD) grows rapidly by fermentation (log phase) and then switches to the utilization of non-fermentable carbon sources (diauxic shift) in the post-diauxic phase (Werner-Washburne, M., Braun, E. L., Crawford, M. E. & Peck, V. M. (1996) Mol. Microbiol. 19, 1159-66). Cells maintained in expired YPD medium or water after the post-diauxic phase decrease metabolism and macromolecular synthesis by more than 100 fold, and survive for months in stationary phase by slowly utilizing reserve nutrients (Werner-Washburne, M., Braun, E. L., Crawford, M. E. & Peck, V. M. (1996) Mol. Microbiol. 19, 1159-66; Lillie, S. H. & Pringle, J. R. (1980) J. Bacteriol. 143, 1384-1394).

Thus, in replicative aging the unicellular Saccharomyces cerevisiae undergoes an age-dependent increase in cell dysfunction and mortality rates (C. E. Finch, Longevity, Senescence, and the Genome (University Press, Chicago, 1990); J. W. Vaupel, et al., Science 280, 855-60 (1998)). Replicative aging in yeast is also associated with an enlargement of the cell and a slowing in the budding rate, and is commonly measured by counting the number of buds generated by a single mother cell (replicative life-span or budding life-span) (N. K. Egilmez, S. M. Jazwinski, J Bacteriol 171, 37-42 (1989); D. Sinclair, K. Mills, L. Guarente, Annu Rev Microbiol 52, 533-60 (1998)). The replicative life span of yeast is regulated by the Sir2 protein, which mediates chromatin silencing in a NAD-dependent manner (D. Sinclair, K. Mills, L. Guarente, Annu Rev Microbiol 52, 533-60 (1998); S. J. Lin, P. A. Defossez, L. Guarente, Science 289, 2126-8. (2000)).

However, yeast can also age chronologically as a population of non-dividing cells (V. D. Longo, Neurobiol Aging 20, 479-86 (1999); J. W. Vaupel, et al., Science 280, 855-60 (1998); D. Sinclair, K. Mills, L. Guarente, Annu Rev Microbiol 52, 533-60 (1998)). S. cerevisiae grown in complete glucose medium (SC) stop dividing after 24 to 48 hours and survive for 5 to 7 days while maintaining high metabolic rates (V. D. Longo, Neurobiol Aging 20, 479-86 (1999); V. D. Longo, L. M. Ellerby, D. E. Bredesen, J. S. Valentine, E. B. Gralla, J. Cell Biol. 137,1581-8 (1997); Lee-Loung Liou, Paola Fabrizio, Vanessa N. Moy, James W. Vaupel, , Joan SelverstoneValentine, Edith Butler Gralla, and Valter D. Longo (Unpublished results)(Lee Loung Liou, Ph.D. Thesis University of California Los Angeles, 1999)). Survival in the post-diauxic and stationary phases is called “chronological life span” to distinguish it from the “budding life span” described above (Sinclair, D., Mills, K. & Guarente, L. (1998) Annu. Rev. Microbiol. 52, 533-60; Jazwinski, S. M. (1996) Science 273, 54-9).

Chronological aging of yeast is a situation more akin to their experience in nature where they are likely to survive as non-dividing populations exposed to scarce nutrients. For this reason, and to avoid extended growth and entry into the hypometabolic stationary phase induced by incubation in the nutrient-richer YPD medium (M. Werner-Washburne, E. L. Braun, M. E. Crawford, V. M. Peck, Mol. Microbiol. 19, 1159-66 (1996)), in accordance with the invention, aging is studied using yeast grown in SC medium.

Mechanisms that regulate chronological aging are poorly understood. Chronological survival in yeast is extended by overexpression of the human oncoprotein Bcl-2 (Longo, V. D., Ellerby, L. M., Bredesen, D. E., Valentine, J. S. & Gralla, E. B. (1997) J. Cell Biol. 137, 1581-8), known to protect mammalian cells against oxidative stress (Kane, D. J., Sarafian, T. A., Anton, R., Hahn, H., Gralla, E. B., Valentine, J. S., Ord, T. & Bredesen, D. E. (1993) Science 262, 1274-7) and is shortened by null mutations in either or both superoxide dismutases (Longo, V. D., Gralla, E. B. & Valentine, J. S. (1996) J. Biol. Chem. 271, 12275-12280).

As the examples below demonstrate, this invention is based on experiments that have greatly expanded the understanding of the mechanism of chronological aging in yeast. Yeast cells mutagenized by transposon insertion or other methods and maintained in the post-diauxic and stationary phase have been used to screen for, and quickly identify, mutations that increase longevity, multiple stress-resistance, or resistance to specific toxins or conditions. Such long-lived mutants are then used to elucidate aging mechanisms to provide methods and compositions for extending longevity and for treating age-related diseases.

Thus, for example, the long-lived mutants can be used to 1) identify drugs that prevent the toxicity of specific toxins or mutagens such as superoxide, paraquat, or iron by co-incubation of the potential drug with the toxin or by using specific yeast mutanst that accumulate toxins in a specific organelle (such as yeast mutants lacking the enzyme superoxide dismutases), and 2) screen for drugs that affect the function of human proteins inserted into yeast cells lacking the yeast homolog of that particular human protein but that do not decrease long-term survival.

One advantage of the invention is the ability to provide an inexpensive and efficient system to quickly identify proteins and drugs that increase stress-resistance and longevity. To screen for similar proteins or drugs in mammalian systems would be much more complex and expensive. As established by the invention and discussed in more detail below (FIG. 15), the regulation of aging and stress-resistance is conserved from yeast to man. Furthermore, the longevity mutations (in yeast) identified in the examples below are in genes whose sequence and function is highly conserved from yeast to man. Thus, it is reasonable to assume that the genes identified or their homologs are involved in longevity extension and the protection of cells against stress and diseases in man.

Specific findings from the experiments of the examples below are summarized below.

Example 1: The example shows that mutations in CYR1 and in SCH9 increase chronological life span of S. cerevisiae. The example also shows that long-lived mutants were also resistant to paraquat and heat shock, establishing that resistance to multiple stresses is associated with increased longevity. Allele rescue of the mutants revealed that transposons had integrated in the promoter region of the Sch9 protein kinase gene for one mutant and in the N-terminal regulatory region of adenylate cyclase of the other mutant. Transformation of the first mutant with wild type SCH9 and of the second mutant with CYR1, abolished the survival extension, strongly suggesting that the decreased expression or activity of Sch9 and Cyr1 extends survival (results not shown). When the SCH9 gene is deleted, sch9Δ mutants grew slowly but survived three times longer than wild type cells. The example also establishes that the protein kinase activity of Sch9 accelerates mortality in non-dividing yeast because transformation of sch9Δ with wild type SCH9 reversed the life-span extension, whereas transformation with the genes encoding for the inactive Sch9_{k441A} or Sch9_{D556R} kinases did not.

Example 2: The example shows that transcription factors Msn2, Msn4 and protein kinase Rim15 are required for chronological life-span extension of certain long-lived yeast mutants. The activation of these factors increases resistance to thermal stress, thus associating resistance to thermal stress with longevity. These transcription factors also function in pathways for inducing the expression of genes encoding for several heat shock proteins, catalase (CTT1), and the DNA damage inducible gene DDR2. Thus, the modulation of these pathways and/or the regulation of the related genes provide avenues for extending the life-span of eukaryotes.

Example 3: This example reinforces the conclusion that long-lived mutants exhibit increased resistance to heat-shock and oxidative stress. Thus, pathways and genes that contribute to increased cellular resistance to heat-shock and/or oxidative stress are targets for increased longevity of an organism or for treating aging-dependent diseases.

Example 4: This example shows that mutations in long-lived mutants delay the reversible inactivation of the superoxide-sensitive enzyme aconitase in the mitochondria. Thus, superoxide toxicity plays a role in chronological aging.

Examples 5-12: Examples 5-12 establish the role of Ras2 and superoxide in the regulation of survival in yeast and, by analogy other eukaryotes. Ras2 was chosen because of its role in regulating antioxidant protection and thermotolerance (Toda, T., Uno, I., Ishikawa, T., Powers, S., Kataoka, T., Broek, D., Cameron, S., Broach, J., Matsumoto, K. & Wigler, M. (1985) Cell 40, 27-36; Belazzi, T., Wagner, A., Wieser, R., Schanz, M., Adam, G., Hartig, A. & Ruis, H. (1991) Embo J. 10, 585-592) and because its constitutive activation decreases survival in non-dividing yeast (Toda, T., Uno, I., Ishikawa, T., Powers, S., Kataoka, T., Broek, D., Cameron, S., Broach, J., Matsumoto, K. & Wigler, M. (1985) Cell 40, 27-36).

The results of the experiments of Example 5-12 show that the expression of mitochondrial SOD2 is required for the longevity extension caused by mutations that decrease the activity of the Ras/cAMP/PKA and Sch9 pathways and that superoxide toxicity plays an important role in yeast aging and death. The results further show that other systems are important for delaying aging and death.

The previous examples had shown that the expression of genes regulated by stress resistance transcription factors and kinases, including Msn2, Msn4, and Rim15, mediates chronological life span extension in yeast [Examples 1-4]. The SOD2 promoter contains an STRE element regulated by Msn2/Msn4 and a PDS element regulated by transcription factor Gis1, which functions downstream of Rim15 [Flattery-O'Brien, 1997 #443; Pedruzzi, 2000 #908]. The reduced life span of ras2Δ, cyr1::mTn, and sch9Δ mutants lacking SOD2 establishes that longevity is extended in part by inducing SOD2 expression. The increase in SOD2 expression in sch9Δ mutants supports this conclusion.

The expression of SOD2 did not increase at day 5 and 6 in cyr1::mtn mutants. This may be because the early down-regulation of mitochondrial respiration in cyr1 mutants may have caused an early decrease in the enzymes that protect mitochondria against oxidative damage, including SOD2. In fact, the levels of SOD2 in cyr1::mtn mutants are slightly higher than in wild type cells during early log phase. The early entry of cyr1::mtn mutants in a hypometabolic state may also provide an explanation for the limited effect of the SOD2 deletion on the extended survival of cyr1::mtn mutants since the 100 fold decrease in respiratory rates should minimize superoxide generation.

The examples show that double overexpression of SOD1SOD2 but not of SOD1 and catalase or of each enzyme alone extends survival by 30%. Although SOD1 is found mainly in the cytosol, it also reaches the mitochondrial intermembrane space [Okado-Matsumoto, 2001 #1015]. Its role may be to protect against the superoxide generated in mitochondria and released into both the matrix and the intermembrane space [Han, 2001 #1014]. Therefore, overexpression of SOD1 may also protect yeast against mitochondrial superoxide and the effect of SOD1SOD2 overexpression on life span extension may be caused by the scavenging of mitochondrial superoxide by both enzymes. However, increased protection against cytosolic superoxide may also be important to lengthen survival.

The association between mortality increase and aconitase reactivation is also consistent with a role for superoxide-dependent mitochondrial damage in yeast aging and death. In fact, aconitase inactivation and reactivation are lower in long-lived mutants than in wild type yeast [Example 3]. The examples also show that iron and sulfur can restore aconitase activity, which indicates that the enzyme is present in the cells in a form that can be reactivated; most likely in a 3Fe-4S cluster form [Gardner, 1995 #96]. The inactivation of aconitase and the consequent release of iron from the 4Fe-4S cluster may further increase oxidative damage by promoting the generation of the strong oxidant hydroxyl radical by Fenton chemistry [Fridovich, 1995 #87].

The examples also provide evidence for the role of superoxide and mitochondrial damage in decreasing survival. This evidence is provided by the effect of paraquat and antimycin A on life span (FIG. 8C). The examples show that both paraquat and antimycin A, which promote the generation of superoxide, are toxic to wild type cells (FIG. 8C) and are particularly toxic to sod2Δ mutants. These results are consistent with finding that mutations in the COQ3 gene, which encodes for a protein involved in mitochondrial coenzyme Q biosynthesis [Poon, 1999 #1017], also cause early death in the post-diauxic phase. The examples establish that high levels of superoxide and the loss of mitochondrial function decrease survival in yeast (FIG. 8C).

The inactivation of aconitase may be a stochastic event caused by aging or may be part of a regulatory mechanism [Gardner, 1995 #96]. For example, by reversibly inactivating aconitase, superoxide could act as a signaling molecule that regulates the TCA cycle and metabolic rates. One possibility is that mitochondrial damage and death occur in yeast cells that are unable to prevent superoxide toxicity when exposed to levels of superoxide normally required for signaling. The observation that yeast cells down-regulate antioxidant enzymes even though these enzymes are important for long-term survival may be explained by the existence of a form of programmed cell death or programmed aging may exist in yeast.

The examples further show that the induction of antioxidant protection can only account for part of the longevity extension caused by mutations in the Ras/cAMP and Sch9 pathways. The effect of the overexpression of both SOD1 and SOD2 on life span is much smaller compared to that caused by ras2Δ or sch9Δ mutations. Furthermore, ras2Δ and cyr1::mtn mutants lacking SOD2 survive shorter than the SOD2 mutants but survive longer than wild type, establishing that other systems contribute to life span extension.

Previous aging studies in yeast have studied the replicative rather than chronological life span. These studies often offer conflicting data. One study demonstrated that the deletion of ras2 slightly increases the replicative life span whereas the constitutive activation of Ras2 caused a drastic decrease in survival [Pichova, 1997 #690]. However another study shows that ras2 null mutants have a decreased replicative life span and cells with increased Ras2 activity have a decreased replicative life span [Sun, 1994 #330].

However, this invention is directed to chronological life span extension, which is associated with increased stress resistance and reduced superoxide toxicity. In fact, stress resistance genes MSN2/MSN4 are required to increase chronological survival but not to extend replicative life span.

The examples show that S. cerevisiae strains SP1 and DBY746 grown in minimal SDC medium and maintained in this medium survive in an alternate high-metabolism state for the majority of the life span (FIG. 11). Under these conditions, these strains maintain high metabolic rates for 96-120 hours and low metabolic rates for an additional 24-48 hours before reaching the mean survival point (FIG. 11).

The findings of the examples can be combined with previous studies to firmly establish yeast as the model organism to study aging in eukaryotes. Previous studies in worms, flies and mice suggest that the insulin/IGF-1, or a related pathway, regulate longevity. The results of our experiments described in the examples below add vastly to the prior understanding of aging as follows:

1) In this invention the entire pathway that regulates longevity in yeast is described: including Ras2, adenylate cyclase, PKA, MSN2/MSN4/RIM15, SOD2. Furthermore, the role of superoxide and specifically of the mitochondrial superoxide dismutase SOD2 in longevity extension is described for the first time. Studies in other organisms identified mutations in certain signal transduction genes but did not describe an entire pathway and did not provide evidence for the molecular mechanisms that mediate longevity regulation by these pathways in these organisms.

2) In mice the role of IGF-1 in longevity regulation has not been demonstrated. Only the role of growth hormone in longevity regulation has been demonstrated and the mechanisms or action are unknown. In flies, an insuli/IGF-1 receptor has been implicated in longevity extension but the downstream mechanisms are unknown. A juvenile hormone that acts downstream of the insulin/IGF-1 receptor has been proposed as the mediator of aging (Kenyon 2001 review, Cell). In worms, a pathway that includes an insulin/IGF-1-like receptor and other genes conserved from worms to humans has been shown to regulate longevity. However, the conclusion from the work in worms is that the insulin/GF-1-like pathway regulates longevity by regulating the generation of a downstream hormone (Kenyon 2001 review, Cell). The insulin/IGF-1-like receptor is believed to regulate the release of a hormone from one cell type, which is then released and reaches other cells in the organism. The conclusion is that the secondary hormone regulates longevity. This invention using yeast as a model organism shows that it is an insulin/IGF-1-like pathway that directly regulates resistance to damage and longevity. There are no secondary hormones involved. Therefore, the work in yeast suggests that the down-regulation of the IGF-1 pathway in all human cells will delay aging and age-related diseases. By contrast the previous work suggest that the down-regulation of the pathway regulated by an unknown hormone will delay aging.

3) This invention provides the first clear evidence for the conservation of longevity regulation in organisms ranging from yeast to humans. Previously, the only longevity regulatory pathway had been identified in worms. However, there was no evidence that a similar or analogous pathway was functioning in other eukaryotes and, therefore, there was no evidence that such a pathway may be present in humans. The studies in yeast presented in this invention show that yeast and worms regulate longevity by very similar mechanisms and by a set of genes conserved from yeast to humans.

Thus, this invention establishes for the first time that there are remarkable similarities between the genes and pathways involved in the regulation of longevity in yeast and worms. The examples show that, in yeast, the down-regulation of glucose signaling by ras2, cyr1 and sch9 mutations increases longevity and resistance to oxidative stress and heat shock. Also, as seen earlier, in the cyr1 mutants chronological life span extension is mediated by stress resistance transcription factors Msn2 and Msn4, which induce the expression of genes encoding for several heat shock proteins, catalase (CTT1), the DNA damage inducible gene DDR2, and SOD2. Analogously, in worms, mutations in the signal transduction genes age-1 and daf-2, extend survival by 65% to 100% [Kenyon, 1993; Johnson, 1990] and increase thermotolerance and antioxidant defenses [Kimura, 1997; Larsen, 1993; Lithgow, 1995], apparently through stress resistance transcription factor DAF-16 [Lin, 1997]. The examples also show that in yeast, chronological life span extension is associated with decreased superoxide generation and aconitase inactivation in the mitochondria. The examples show that SODs are required for life span extension in ras2, cyr1, and sch9 mutants and that the overexpression of superoxide dismutases extends longevity. In worms, among the genes regulated by the daf-2 pathway are several heat shock proteins and mitochondrial MnSOD [Honda, 1999; Cherkasova, 2000]. It is also known that the yeast Ras/Cyr1/PKA pathway down-regulates glycogen storage and genes involved in the switch to the hypometabolic stationary phase and to the dormant spore state [Boy-Marcotte, 1998; Werner-Washburne, 1996]. The worm daf-2 pathway also regulates the storage of reserve nutrients (fat and glycogen) and the switch to the hypometabolic dauer larvae state [Kenyon, 1993; Morris, 1996; Kimura, 1997]. Thus, in addition to the high sequence similarities between the yeast SCH9 and the worm AKT-1/AKT-2 serine/threonine kinase genes, the examples show that these two unrelated organisms regulate stress resistance and longevity by modulating the activity of similar proteins and pathways. Therefore, for the first time, the invention provides evidence for the conservation of aging and longevity mechanisms between two very different eukaryotes.

Considering that yeast and worms are separated by hundreds of millions of years of evolution and that insulin/IGF-1-like pathways have recently been implicated in the regulation of longevity in flies and mice, this invention provides strong evidence for the conservation of longevity in many if not all eukaryotes. Notably, Ras, Sch9/AKT, and SOD2 are highly conserved and function in the yeast longevity regulatory pathway and in the human insulin/IGF-1 pathway, but only either AKT or/and an IGF-1 receptor have been shown to function in the worm or fly longevity pathways. Based on the foregoing a model of aging mechanisms can be provided for organisms ranging from yeast to mice. Such a model is shown FIG. 15. Clearly, the remarkable conservation of genes and pathways that regulate longevity in these unrelated organisms strongly suggests that cellular damage and longevity is also regulated by a pathway that includes IGF-1/Ras/AKT/SOD2 in humans.

The examples show that SOD2 and mitochondrial superoxide play important roles in the aging and death of yeast and establish that the constitutive induction of multiple protection systems can extend longevity. The examples implicate the role of mitochondrial superoxide and aconitase inactivation in the senescence of higher eukaryotes.

### EXAMPLES

### Materials and Methods

### Viability

Viability, which is defined as the ability of a single organism to reproduce and form a colony within 48 hours (Colony Forming Units or CFU) was measured by a live/dead fluorescent assay following the manufacurer's instructions for stationary phase cells (Molecular Probes). The loss of CFUs was also compared to the concentration of proteins in the medium, which correlates with increased cell damage and lysis. Using these two methods, we determined that cell death followed loss of the ability to form a colony (CFU) by 5 to 7 days.

### Strains for Examples 1-4

Strain DBY746 was used in all these examples: MATIleu 2-3, 112 his3Δ1 trp1-289 ura 3-52 GAL⁺. All other strains are isogenic derivatives of DBY746 and were generated in this study. SCH9 disruption was made by using the BamH1 fragment of plasmid psch9.19::URA3 provided by Hirsh, J. MSN2 was disrupted by integration of the Sall fragment of plasmid pt32-DXB::HIS3 provided by Carlson, M. MSN4 and RIM15 deletions were constructed respectively using the HindIII-BamHI fragment of plasmid pAS20 and the XhoI-SacII fragment of plasmid pSV117 provided by Garrett, S. and Mitchell, A. All the deletions were tested by Southern Blot Analysis or PCR Analysis. Low copy plasmids pRS416-HA3-Sch9, (HA). pRS416-HA₃-Sch9_{K441A}, and pRS416-HA₃-Sch9_{D556R} were provided by Thiele, D J. Plasmid pEFCYR1 overexpressing the CYR1 gene was provided by Field J.

### Strains for Examples 5-12

Table II lists the strains used in the experiments of Examples 5-12. Strains lacking RAS2, SOD2, and MSN2/MSN4 were produced by one-step gene replacement using disruption plasmids pRAS2::LEU2 [Kataoka, 1984], pSOD2::TRP1 [Gralla, 1991], pt32-ΔXB::HIS3 [Estruch, 1993 #889], and pAS26 [Smith, 1998]. All disruptions were verified by PCR analysis or Southern Blot. Overexpressor plasmids were constructed in multicopy vectors YEp351 and YEp352 as follows: YEp351-CTT1 was constructed by inserting a 3.9 kb BamH1-HindIII fragment containing the CTT1 gene into the SaII site of YEp351 using a Sall polylinker. YEp351-SOD2 provided by D. Kosman, contains a 2 kb genomic BamH1 fragment inserted into YEp351. YEp352-SOD1 was constructed by ligating a 2 kb SOD1 SphI fragment into the SphI site of YEp352. All the genes described above are driven by their natural promoters. These plasmids were used to construct strains overexpressing CTT1, SOD1, SOD2, alone and in combinations, in both the SP1 and DBY746 backgrounds.

All DNA and RNA manipulations were performed using standard techniques. Yeast transformants were obtained by lithium acetate method [Gietz, 1992].

### Media, Growth Conditions, and Post-diauxic Phase Survival

Unless stated otherwise, all experiments were performed in liquid media in SDC-synthetic complete medium with 2% glucose, supplemented with amino acids, adenine, uracil as well as a four-fold excess of the supplements tryptophan, leucine, histidine, lysine and methionine. Overnight cultures were grown in selective media and inoculated into flasks with a flask volume/medium volume ratio of 5:1 and grown at 30° C. with shaking at 220 rpm. Maximum population density is normally reached after 72 hours of growth in SDC medium. The maximum size of the viable population was approximately 100 million cells/ml, for a total of 5 billion organisms in each flask.

To determine the number of viable yeast, one or two 10 microliter aliquots were removed from each flask and serially diluted. Each aliquot was then plated twice onto YPD plates for a total of 2 or 4 platings/population/day. Serial dilutions were performed in order to plate approximately 100 viable organisms per plate. Viability is defined as the ability of a single organism to reproduce and form a colony within 48 hours (Colony Forming Units or CFU). Viability was also measured by a live/dead fluorescent assay following the manufacurer's instructions for stationary phase cells (Molecular Probes). The percentage of metabolically active (red fluorescence) cells was determined at various points of the life span and was adjusted by taking into account the number of cells that had lysed since the beginning of the experiment. That number of cells that has lysed during the study was determined by light microscopy count of all intact. The loss of CFUs was also compared to concentration of proteins in the medium, which should correlate with increased cell damage and lysis.

The significance of the difference between the survival of different strains was calculated by Two-Factor ANOVA analysis with replication using Microsoft Excel.

### Northern Analysis

RNA filters were prehybridized with 100 μg/ml of salmon sperm DNA at 42° C. for 3 hours in buffer containing 1% SDS, 50% formamide, 5×SSC, 5×Denhardt's solution and then incubated overnight with a ³²P-labeled 2 kb BamHI SOD2 fragment. After hybridization the filters were washed in the following manner: twice in 2×SSC, 0.1% SDS (2 min and 5 min) at 42° C., and twice in 0.1×SSC, 0.1% SDS (10 min and 30 min) at 60° C. The filters were exposed, developed, and scanned using the PhophorImager system (Molecular Dynamics).

### Oxygen Consumption

Cellular oxygen uptake was measured at 30° C. in a 4 ml stirred chamber using a YSI MODEL 53 Biological Oxygen Monitor (Yellow Springs Instruments) following the manufacturers directions. Cells were cultured as described above, except they were inoculated at an initial density of 1×10⁶ cells/ml and incubated for the indicated time before aliquots were removed and tested for oxygen consumption. Cells were kept in the medium in which they had been growing, and conditions that resembled the flask environment (30° C. and stirring) were maintained in the chamber.

### Index of Respiratoy Competence (IRC) Measurement

Yeast cells were incubated in SDC medium and switched to water on day 3. Aliquots were removed from the cultures every 2-3 days, serially diluted, and plated onto YPD and YPG (3% glycerol as carbon source) plates. The latter medium requires respiratory competence for the yeast in order to grow. Therefore, viability on YPG, which is measured as percentage of the viability in YPD, is defined as IRC.

### SOD and Catalase Activity Assays

Superoxide dismuatase assays were performed by using the method of auto-oxidation of 6-hydroxydopamine [Heikkila, 1976 #134]. For separate measurement of CuZnSOD and MnSOD, inhibitors were used to inhibit or inactivate the respective enzyme, and the individual activities were calculated accordingly [Geller, 1984 #1041]. To determine MnSOD activity, 1 mM KCN, which inhibits 95% of the CuZnSOD activity, was added to the mix. To measure the CuZnSOD activity, extracts were treated with 2% SDS for 1 hour at 37° C. to inactivate MnSOD, the SDS was removed by incubating with 0.3 M KCl for 30 min at 4° C., centrifuged at 20,000 g for 10 min, and the extracts were assayed as described above. Catalase activity was determined by monitoring the disappearance of hydrogen peroxide spectrophotometrically at 240 nm in 50 mM potassium phosphate buffer, pH 7.0 at 25° C.

### Aconitase Activity and Reactivation

Cells were inoculated at an OD600 of 0.1 in SDC medium and harvested at the indicated times. Whole cell extracts were obtained by glass bead lysis under argon in 50 mM Tris 7.2, 150 mM NaCl, 5 mM EDTA, and 0.2 mM PMSF with an equal volume of 0.5 mm acid washed glass beads, and vortexing for 6 cycles of 30 seconds followed by 2 minutes of cooling. After centrifugation, the supernatants were aliquoted, flash frozen, and stored at −70° C. Because of the instability of 4Fe4S clusters in air, the extraction procedures were performed as rapidly as possible, under an inert atmosphere (argon). Furthermore, aliquots kept at −70° C. were only thawed immediately before the assay. Aconitase activity was measured spectrophotometrically as described (S. Melov, et al., Science 289, 1567-9 (2000)). Briefly, the linear absorbance change at 240 nm (cis-aconitate disappearance) was followed in a reaction mixture containing 1 mM cis-aconitate, 0.5 M NaCl, and 0.1 M Tris 7.4. For iron-sulfur cluster reactivation experiments, 1 mM ferric sulfate and 1 mM sodium sulfide (Na₂S) was added to the cuvette containing all the reagents required for the aconitase assay. Activity was measured as described above.

### Example 1

### Transposon Mutagenesis and Isolation of Long Lived Mutants

To understand the molecular mechanism that regulates yeast longevity, yeast cells were transposon-mutagenized and long-lived mutants isolated (Transposon mutagenesis and allele rescue were performed with the yeast insertion library provided by M. Snyder as described by Ross-Macdonald, P et al, Methods Enzymol., 303, 512-532 (1999)). We screened for mutants that survived both a 1-hour heat stress at 52° C. and a 9-day treatment with the superoxide-generating agent paraquat (1 mM), because of the association between stress resistance and longevity in higher eukaryotes. From 2 billion cells screened, we isolated 4,000 thermotolerant colonies and 40 paraquat-resistant colonies carrying transposons. Out of the 4040 stress-resistant mutants 9 were able to survive to day 9, when most of the wild type cells are dead. The only two mutants isolated independently in both the paraquat and heat shock selections, designated Tn3-5 and Tn3-24, were also the longest-lived (FIG. 6A), establishing that resistance to multiple stresses is associated with increased longevity. Allele rescue of the mutants revealed that transposons had integrated in the promoter region of the Sch9 protein kinase gene (sch9::mTn) (Tn3-5) (33 bp upstream of the start codon) and in the N-terminal regulatory region of adenylate cyclase (cyr1::mTn) (Tn3-24) (between codon 208 and 209). The mean life spans of sch9::mTn and cyr1::mtn were extended by 30% and 90%, respectively. Transformation of Tn3-5 cells with wild type SCH9 and of Tn3-24 cells with CYR1, abolished the survival extension, strongly suggesting that the decreased expression or activity of Sch9 and Cyr1 extends survival (not shown).

To investigate further the role of SCH9 in chronological survival we deleted the SCH9 gene (Supplementary material is available at Science Online at www.sciencemag.org/feature/data/1059497.shl). sch9Δ mutants grew slowly but survived three times longer than wild type cells (FIG. 6B). To determine whether the protein kinase activity of Sch9 accelerates mortality in non-dividing yeast, we transformed mutants with either wild type SCH9 or with forms of SCH9 bearing kinase-inactivating mutations SCh9_{k441A} and sch⁹_{D556R} (K. A. Morano, D. J. Thiele, Embo J 18, 5953-62 (1999)). Transformation of sch9Δ with wild type SCH9 reversed the life-span extension, whereas transformation with the genes encoding for the inactive Sch⁹_{k441A} or Sch9_{D556R} kinases did not (FIG. 6C).

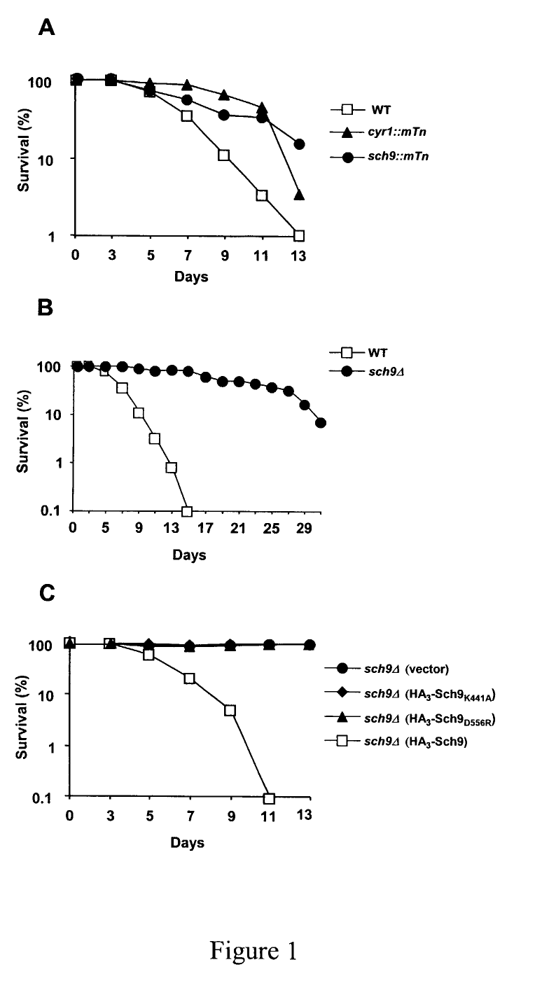

This example shows that mutations in CYR1 and in SCH9 increase chronological life span of S. cerevisiae. The results are shown in FIG. 1 as follows: FIG. 1A: survival of wild type (DBY746), and transposon mutagenized cyr1::mTn (Tn3-24), and sch9::mTn (Tn3-5), FIG. 1B : survival of wild type and sch9Δ, FIG. 1C: survival of sch9Δtransformed with wild type SCH9 or with a mutated sch9 encoding for a catalytically inactive proteins (Sch9_{K441A}, Sch9_{D556R}). Cell viability was measured every 2 days starting at day 3 (Supplementary material is available at Science Online at www.sciencemag.org/feature/data/1059497.Shl ). Experiments were repeated between 3 and 7 times with two or more samples/experiment with similar results. The average of all experiments is shown. The mean life span increase in cyr1::mTn (90%), sch9::mTn (30%), and sch9Δ (300%) significant (P<0.05, ANOVA analysis).

### Example 2

### Molecules and Mechanism that Mediate Survival Extension in Long-Lived Mutants

Both Sch9 and Cyr1 function in pathways that mediate glucose-dependent signaling, stimulate growth and glycolysis, and decrease stress resistance, glycogen accumulation, and gluconeogenesis (J. M. Thevelein, J. H. de Winde, Mol Microbiol 33, 904-18 (1999)). The C-terminal region of Sch9 is highly homologous to the AGC family of serine/threonine kinases which includes Akt/PKB, whereas the N-terminal region contains a C2 phospholipid and calcium-binding motif. The 327 amino acid serine/threonine kinase domain of yeast Sch9 is, respectively, 47% and 45% identical to that of C. elegans AKT-2 and AKT-1, which function downstream of the insulin-receptor homolog DAF-2 in a longevity/diapause regulatory pathway (Supplementary material is available at Science Online at www.sciencemag.org/feature/data/1059497.Shl ; L. Guarente, C. Kenyon, Nature 408, 255-62. (2000); S. Paradis, M. Ailion, A. Toker, J. H. Thomas, G. Ruvkun, Genes Dev 13, 1438-52 (1999)). In this domain conserved from yeast to mammals, Sch9 is also 49% identical to human AKT-1/AKT-2/PKB implicated in biological functions including insulin signaling, the translocation of glucose transporter, apoptosis, and cellular proliferation (E. S. Kandel, N. Hay, Exp Cell Res 253, 210-29 (1999)).

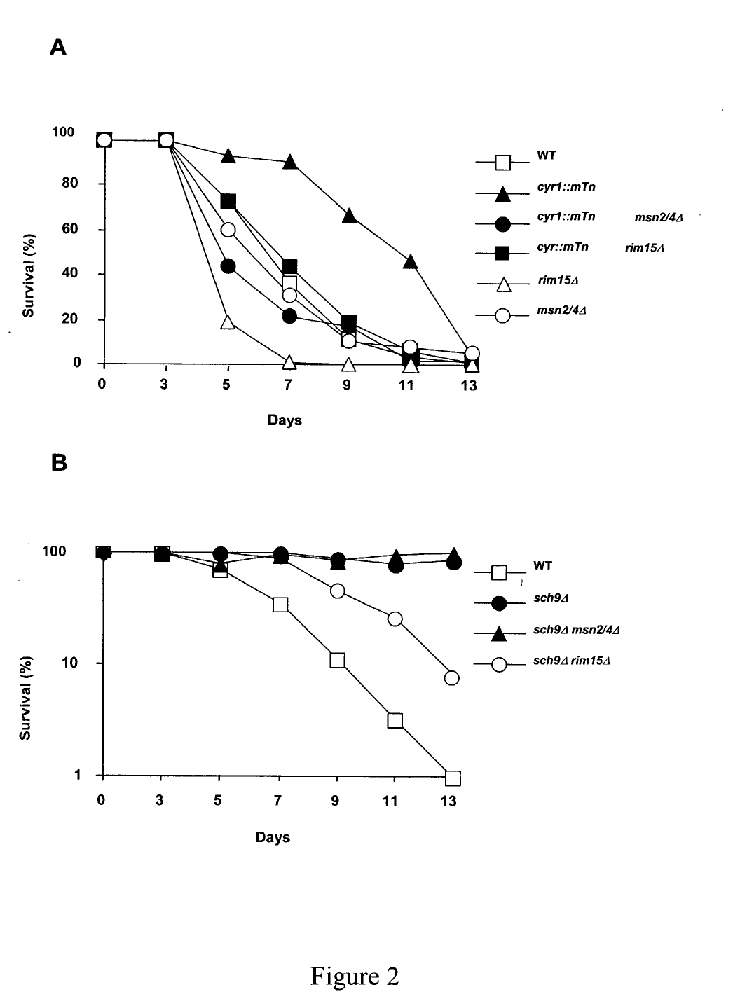

The CYR1 gene encodes for adenylate cyclase, which stimulates cAMP-dependent protein kinase (PKA) activity required for cell cycle progression and growth. The catalytic subunits of PKA are also 35-42% identical to C. elegans and human AKT-1/AKT-2, although PKA belongs to a different family of serine/threonine kinase. The inactivation of the Ras/cAMP/PKA pathway in S. cerevisiae increases resistance to thermal stress in part by activating transcription factors Msn2 and Msn4, which induce the expression of genes encoding for several heat shock proteins, catalase (CTT1), and the DNA damage inducible gene DDR2 (Supplementary material is available at Science Online at www.sciencemag.org/feature/data/1059497.Shl ; J. M. Thevelein, J. H. de Winde, Mol Microbiol 33, 904-18 (1999)). MnSOD also appears to be regulated in a similar manner (J. A. Flattery-O'Brien, C. M. Grant, I. W. Dawes, Mol. Microbiol. 23, 303-12 (1997)). To determine whether MSN2/MSN4 mediate survival extension, we deleted both genes in the cyr1::mTn mutants. The absence of both transcription factors abolished the life-span extension conferred by cyr1::mTn but did not affect the survival of wild type cells (FIG. 2A). By contrast, the deletion of MSN2/MSN4 did not reverse the survival extension in sch9Δ cells (FIG. 2B).

The protein kinase Rim15 regulates genes containing a PDS element T(T/A)AG₃AT involved in the induction of thermotolerance and starvation resistance by a Msn2/Msn4-independent mechanisms (Pedruzzi, N. Burckert, P. Egger, C. De Virgilio, Embo J 19, 2569-79 (2000)). To test the role of Rim15 in survival we generated sch9Δ rim15Δ mutants. The life span of the double mutant was decreased compared to sch9Δ (FIG. 2B). The deletion of RIM15 also abolished the life span extension in cyr1::mtn cells (FIG. 2A). However, it is difficult to establish whether Rim15 mediates the survival extension in these mutants since rim15 single mutants are short-lived (FIG. 2A).

This example establishes that transcription factors Msn2, Msn4 and protein kinase Rim15 are required for the chronological life-span extension of cyr1::mTn and sch9Δ mutants. Results are provided in FIG. 2 as follows: FIG. 2A: Survival of wild type and cyr1::mTn mutants lacking either the stress-resistance genes MSN2/MSN4 or RIM15, FIG. 2B: Survival of wild type and sch9Δ mutants lacking either MSN2/MSN4 or RIM15. Experiments were repeated between 3 and 7 times with two or more samples/experiment with similar results. The average of all experiments is shown.

### Example 3

### Stress Resistance of Long-Lived Mutants

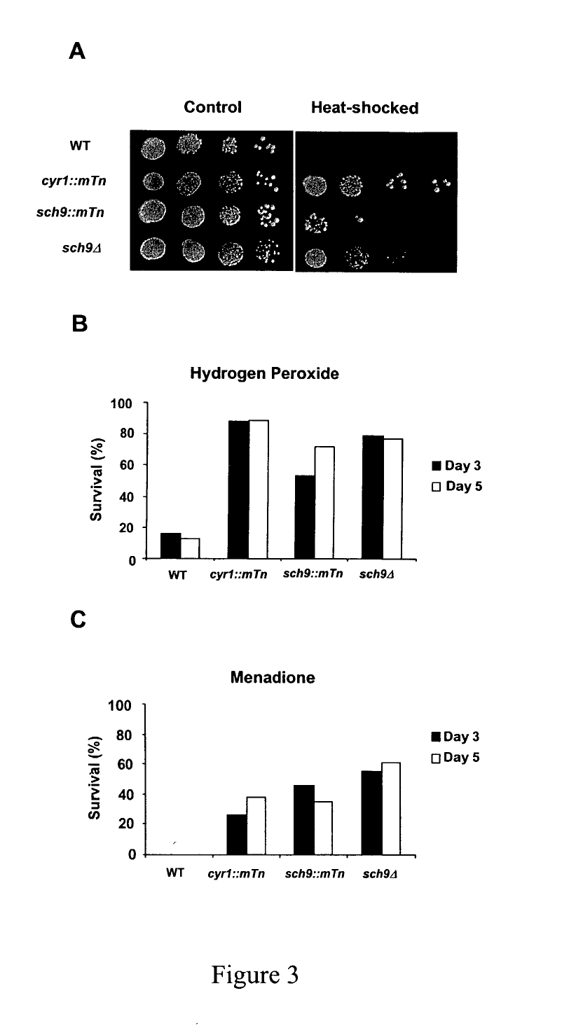

To test whether the long-lived strains were stress-resistant we exposed the mutants to hydrogen peroxide, menadione, or heat. All mutants were resistant to a 1-hour heat shock treatment at 55° C. (FIG. 3A). Similarly, 3-5 day old mutants were resistant to a 30-minute treatment with 100 mM hydrogen peroxide (FIG. 3B) or with the superoxide/H₂O₂-generating agent menadione (20 μM) (FIG. 3C).

This example shows that heat-shock and oxidative stress resistance are increased in long-lived mutants. Serial dilutions (1:1 to 1:1000, left to right) of cells removed from day 1 post-diauxic phase cultures were spotted onto YPD plates and incubated at 30° C. (control) or 55° C. (heat-shocked) for one hour. Pictures were taken after a 4-day incubation at 30° C. The experiment was performed twice with two or more samples/experiment with similar results. The results are shown in FIG. 3A.

Cells removed from days 3 or 5 in the post-diauxic phase were (a) diluted to an OD₆₀₀ of 1 in expired medium and incubated with hydrogen peroxide (100 mM) for 30 minutes or (b) diluted to an OD₆₀₀ of 0.1 in potassium phosphate buffer and treated with 20 μM of the superoxide/H₂O₂-generating agent menadione for 60 minutes. Viability was measured by plating cells onto YPD plates after the treatment. The experiments were performed twice with similar results. The average of the two experiments is shown. The results for (a) and (b) are shown in FIGS. 3B and 3C, respectively.

### Example 4

### The Role of Mitochondrial Aconitase Activity in Long-Lived Mutants

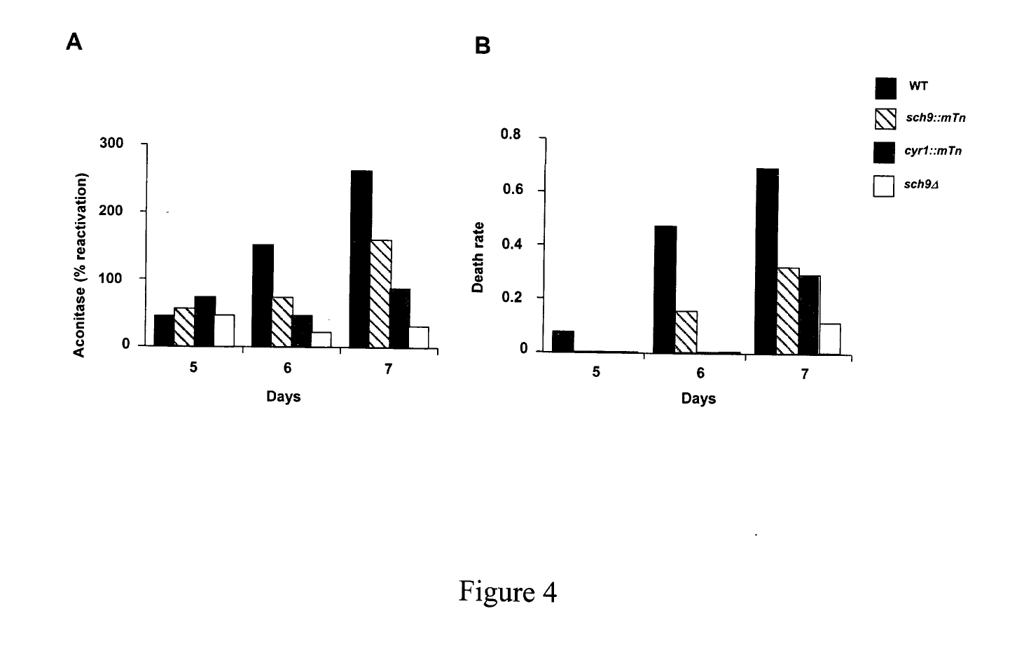

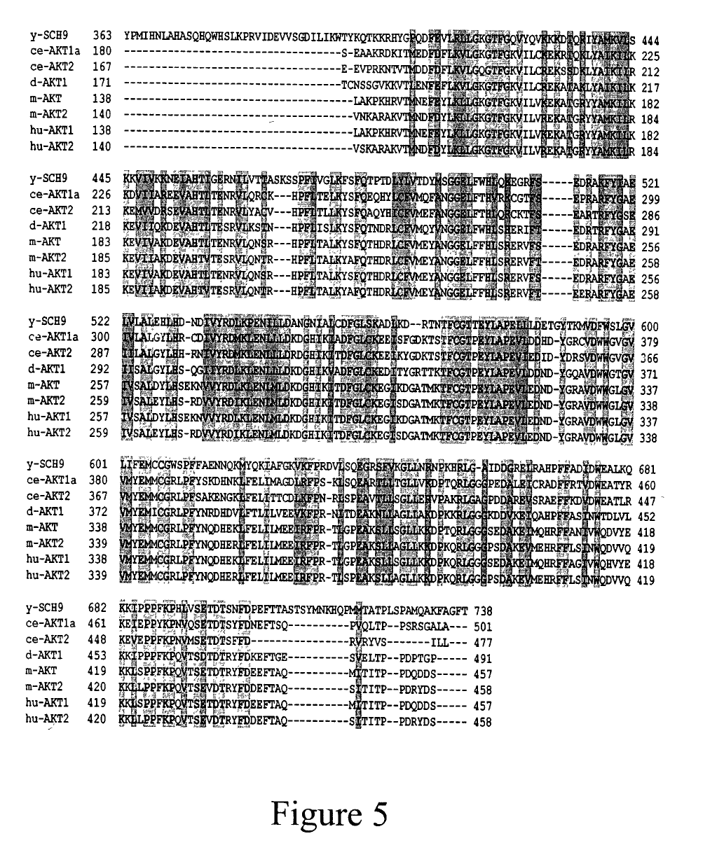

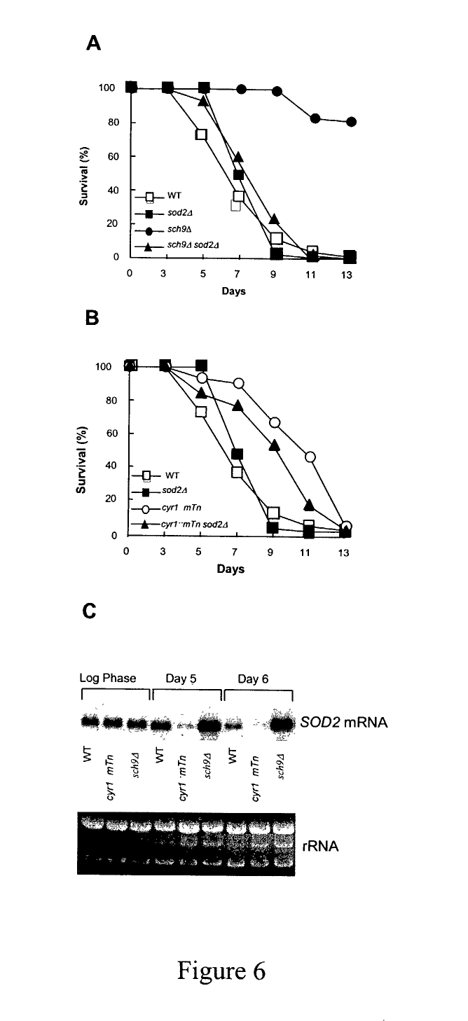

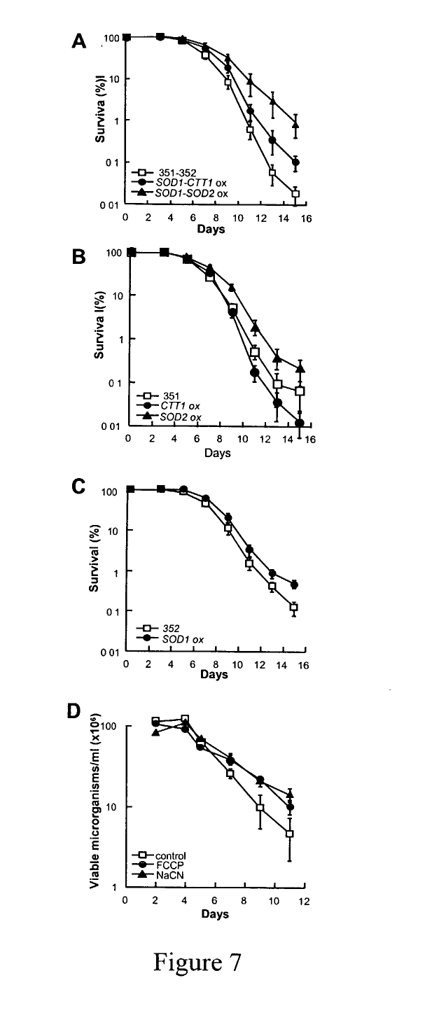

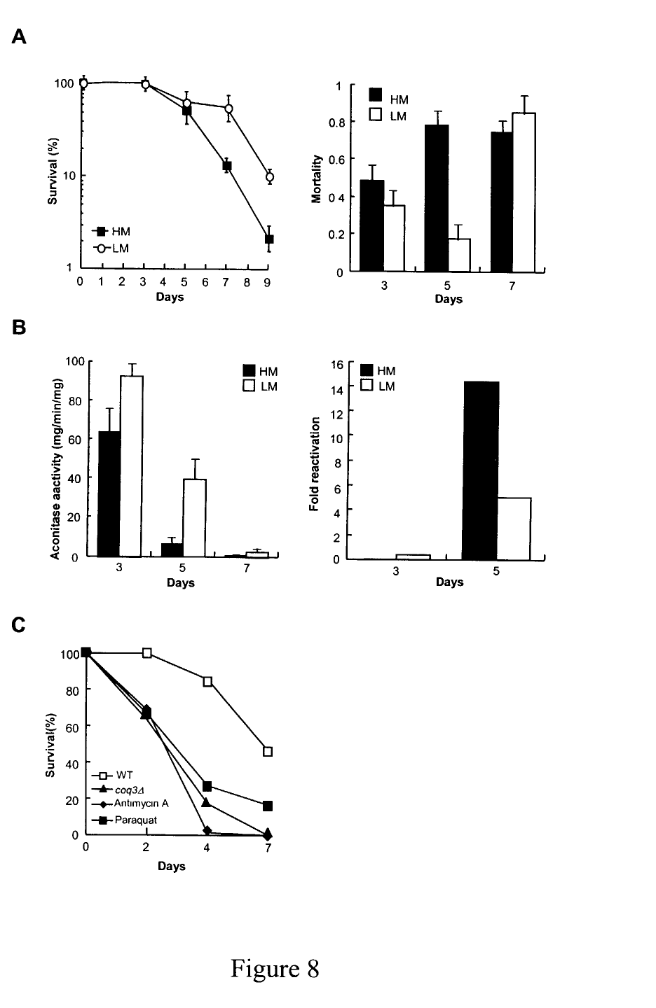

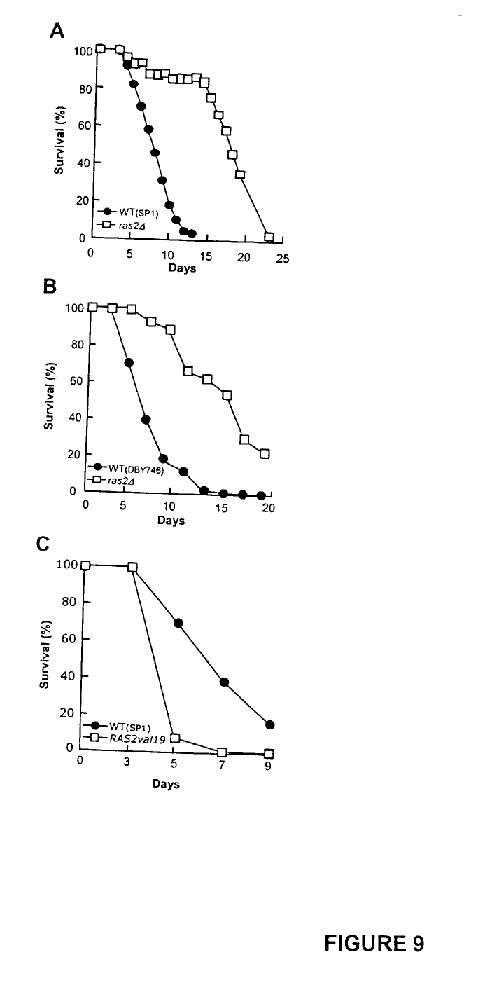

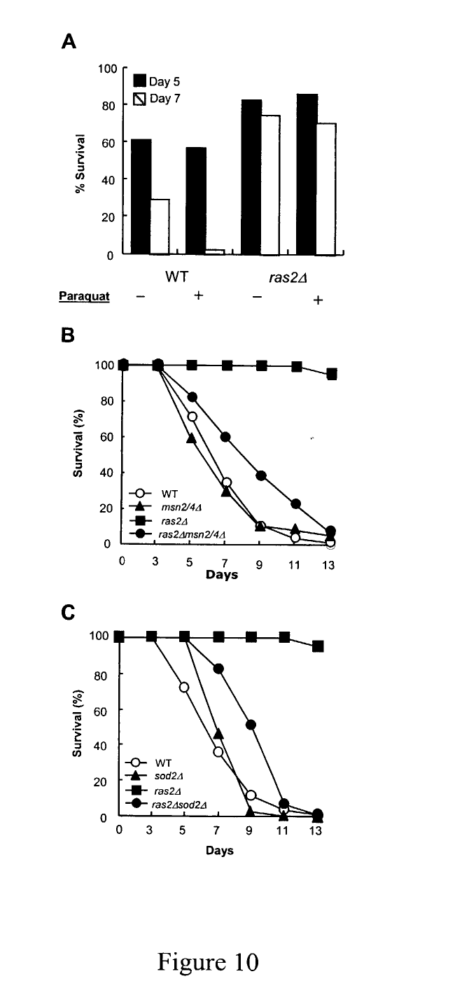

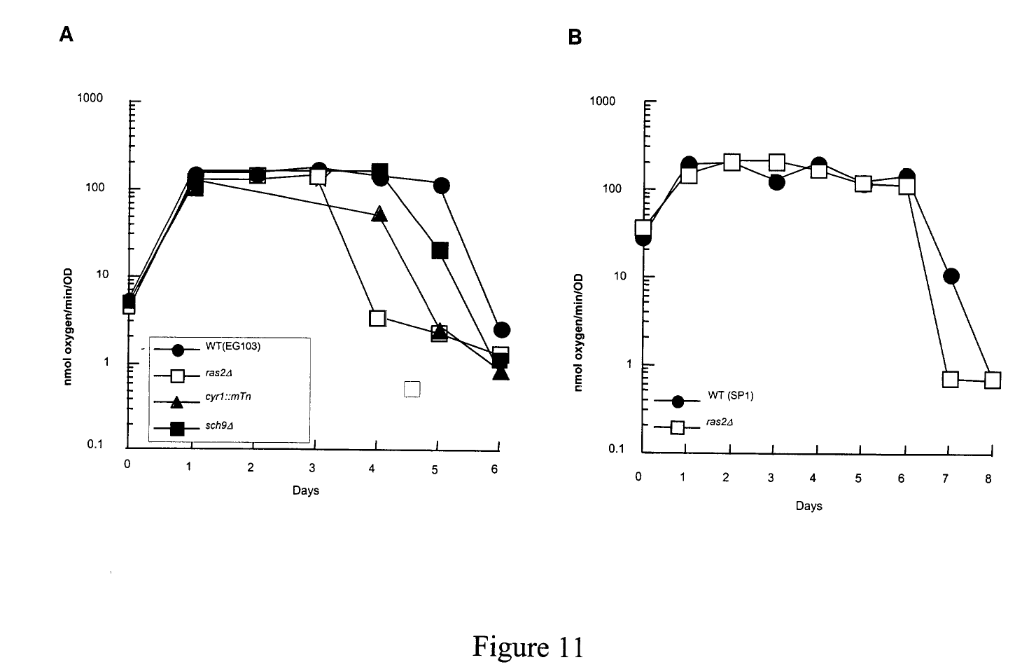

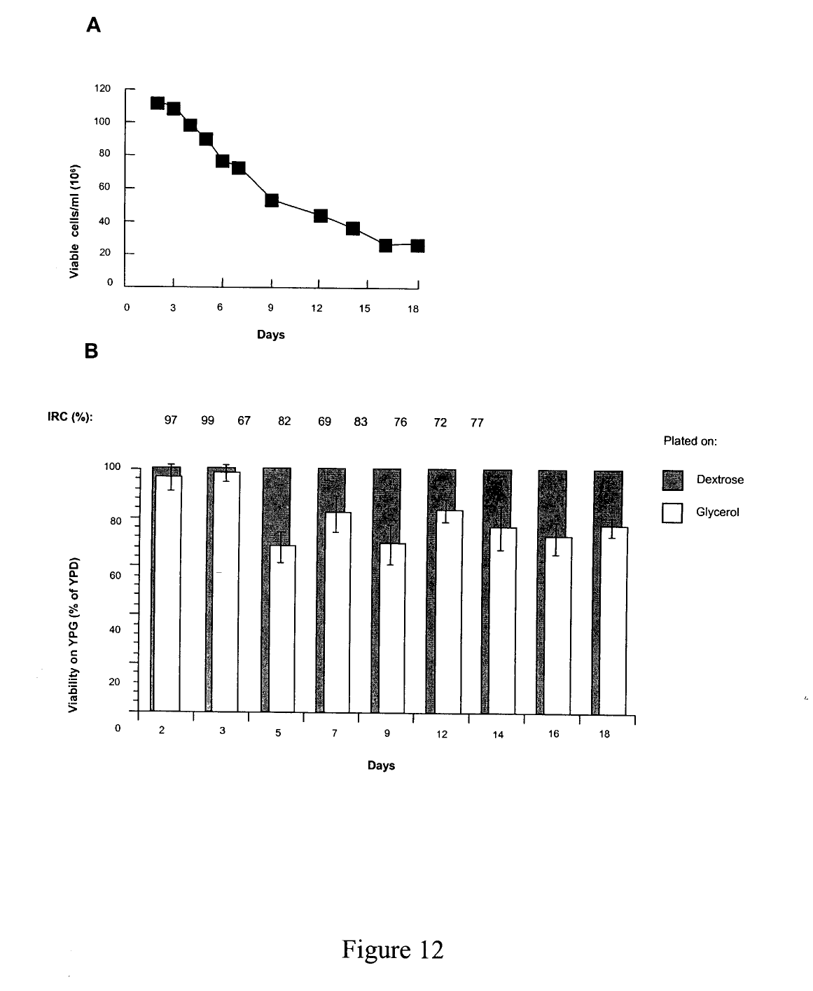

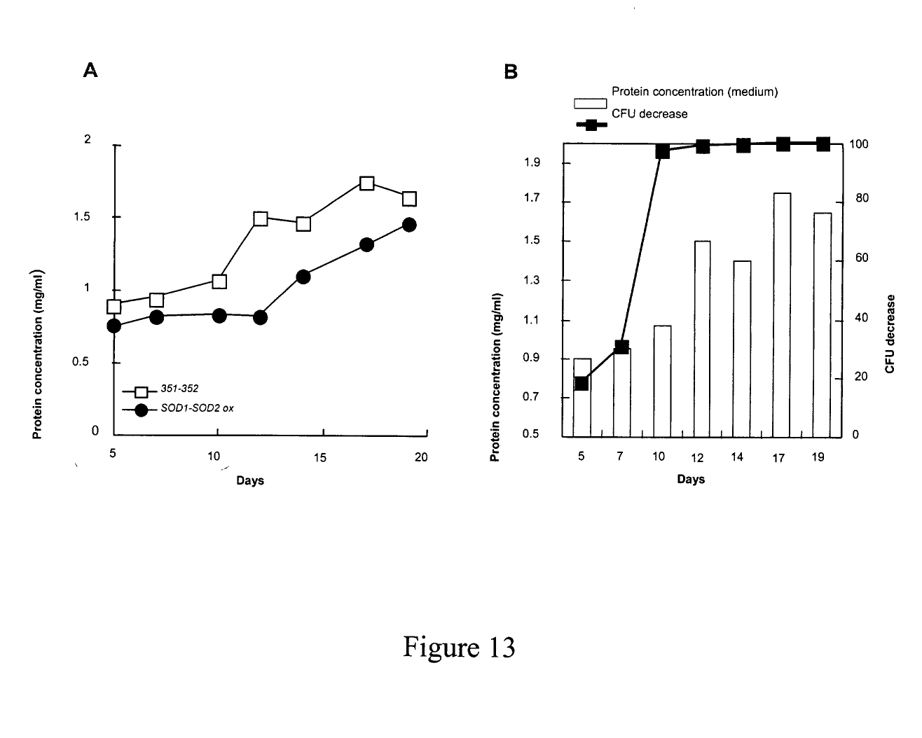

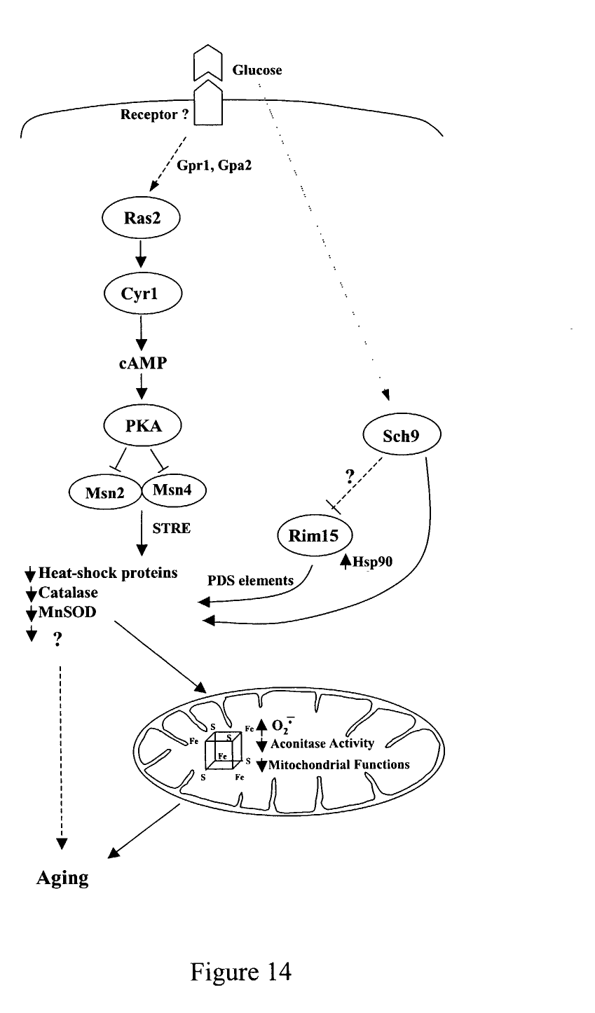

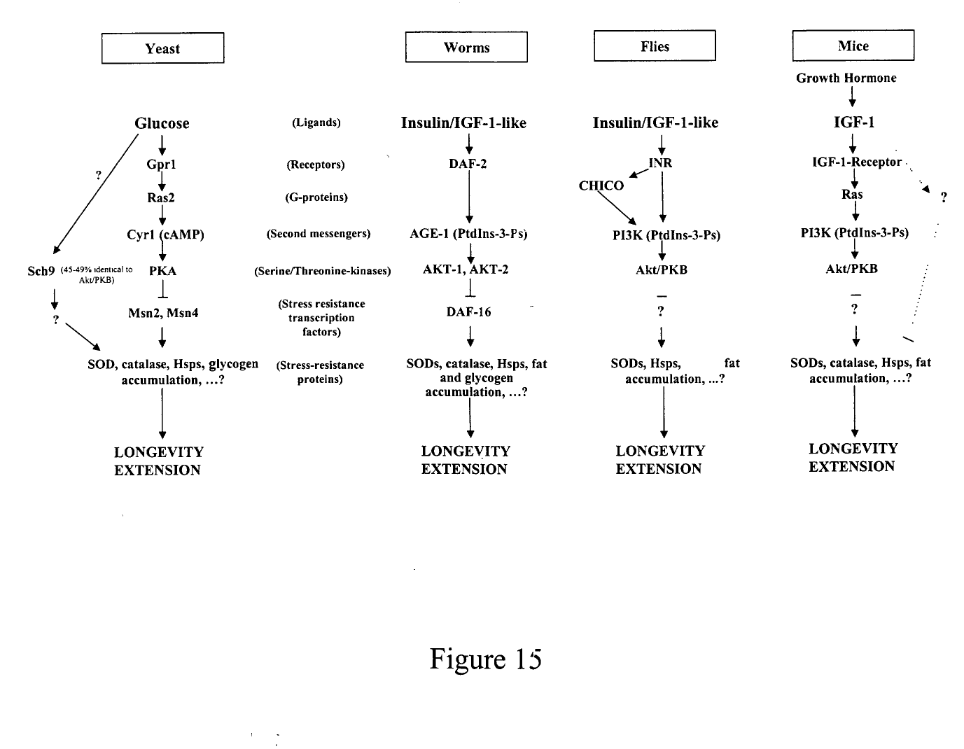

In yeast sod2Δ mutants, superoxide specifically inactivates aconitase and other 4Fe-4S cluster enzymes and causes the loss of mitochondrial function and cell death (V. D. Longo, E. B. Gralla, J. S. Valentine, J. Biol. Chem. 271, 12275-12280 (1996); V. D. Longo, L. L. Liou, J. S. Valentine, E. B. Gralla, Arch. Biochem. Biophys. 365, 131-142 (1999)). To investigate further the role of superoxide toxicity in aging, we monitored the activity and reactivation of mitochondrial aconitase, which can also serve as an indirect measure of superoxide concentration (P. R. Gardner, I. Fridovich, J Biol Chem 267, 8757-63 (1992)). In agreement with the pattern of resistance to superoxide toxicity (FIG. 3C), aconitase specific activity decreased by 50% in wild type cells, and by 30% in cyr1::mtn mutants, but did not decrease in sch9::mTn and sch9Δ mutants at day 7 compared to day 3 (Supplementary material is available at Science Online at www.sciencemag.org/feature/data/1059497.Shl ). The percent reactivation of aconitase was the lowest in the long-lived sch9Δ mutants and the highest in wild type cells (FIG. 4A) and correlated with death rates (FIG. 4B), suggesting that cyr1 and sch9 mutants increase survival, in part, by preventing superoxide toxicity. However, the overexpression of both SOD1 and SOD2 only increases survival by 30% (Lee-Loung Liou, Paola Fabrizio, Vanessa N. Moy, James W. Vaupel, , Joan SelverstoneValentine, Edith Butler Gralla, and Valter D. Longo (Unpublished results)(Lee Loung Liou, Ph.D. Thesis University of California Los Angeles, 1999)), indicating that additional systems, regulated by Msn2, Msn4, and Rim15, are responsible for the major portion of chronological life span extension in cyr1::mtn and sch9Δ mutants.

This example shows that mutations in cyr1 and sch9 delay the reversible inactivation of the superoxide-sensitive enzyme aconitase in the mitochondria. The results are shown in FIG. 4A: Mitochondrial aconitase percent reactivation after treatment of whole cell extracts of yeast removed from day 5-7 cultures with agents (iron and Na₂S) able to reactivate superoxide inactivated 4Fe-4S clusters; and FIG. 4B: Death rate reported as the fraction of cells that lose viability in the 24-hour period following the indicated day.

### Example 5

### The Role of SOD2 in Life Span Extension.

Transcription factors Msn2/Msn4 and Gis1, the latter regulated by Rim15, can activate a variety of stress resistance genes through either a STRE or a PDS element. Among the promoters containing both a STRE and a PDS element is that of SOD2. Thus, SOD2 may function downstream of stress resistance transcription factors Msn2/Msn4 and Gis1 to extend longevity. To test this hypothesis we deleted SOD2 in the cyr1::mtn (PF101) and sch9Δ (PF103) strains. sod2Δ and sch9Δsod2Δ double mutants (PF104 and PF108) survived similarly to wild type cells suggesting that SOD2 is required for the three-fold longer life span of sch9Δ mutants but not for the normal chronological life span of wild type yeast (FIG. 6A). The deletion of SOD2 did not abolish but only reduced life span extension in cyr1::mTn mutants (FIG. 6B, p<0.05). Double sod1Δsod2Δ mutants were not studied since the deletion of both SODs causes a major decrease in life span. The viability for each strain is reported as percent of the viability on day 3 for the same strain.

Between day 3 and 5 almost all the sod2Δ yeast remain viable. Notably, when the survival experiments were performed in 250 ml flasks, instead of the 50 ml flasks used in this study, sod2Δ mutants lost 20-40% of the viability by day 3. Although, the flask volume/medium volume ratio of 5:1 is maintained in both large and small flasks, the larger flask appears to increase the oxygen levels to which cells are exposed and may therefore cause early death in the oxygen-sensitive sod2Δ mutants.

To determine whether the cyr1::mTn and sch9Δ mutations affect the expression of SOD2 we monitored the age-dependent levels of SOD2 mRNA in these mutants. The deletion of SCH9 but not the cyr1::mTn mutation caused a major age-dependent induction of SOD2, as determined by northern blot analysis (FIG. 6C). SOD2 expression in sch9Δ mutants was 3.5 and 8 fold higher than in wild type cells at days five and six, respectively. The low levels of SOD2 mRNA in cyr1::mtn mutants may be explained by the early decrease in oxygen consumption rates in these mutants (FIG. 11), since the expression of the mitochondrial SOD2 should decrease with the decrease in metabolic rates.

### Example 6

### Mitochondrial Superoxide and Survival

To test further the role of superoxide dismutases in the survival extension of cyr1::mTn and sch9Δ mutants (FIG. 6) we measured the chronological life span of yeast overexpressing antioxidant enzymes. We overexpressed various combinations of cytosolic CuZnSOD (SOD1), mitochondrial MnSOD (SOD2), and cytosolic catalase T (CTT1) in wild type strains DBY746 and SP1. The activity of both SOD1 and SOD2 increased by more than 3 fold in SOD1SOD2 overexpressors compared to yeast transformed with plasmid controls (Table III). The activity of catalase also increased by 3-fold in catalase overexpressors (Table III). The overexpression of SOD1 and SOD2 together had the greatest effect on survival (FIG. 7A). The mean chronological life span for SOD1SOD2 double overexpressors in the DBY746 background was increased by 33%, from 6 to 8 days (p<0.05). Double overexpression of SOD1 and CTT1 resulted in a 10% increase in life span (FIG. 7A)(p<0.05). The overexpression of either SOD1 or SOD2 alone resulted in only minor increases in mean survival whereas the overexpression of cytosolic catalase alone slightly decreased survival (FIG. 2B,C). CuZnSOD, MnSOD, and catalase T were also overexpressed in the SP1 background. The overexpression of both SOD1 and SOD2 resulted in a modest, but significant life span extension in this background, with an increase of 10% in mean survival compared to control strains (p<0.05) (data not shown). Single overexpression of either SOD1 or SOD2 in SP1 did not cause a significant improvement in survival (data not shown). The role of mitochondrial superoxide in promoting loss of viability in the post-diauxic phase was confirmed by treating wild type cells with FCCP and NaCN, respectively an uncoupler and inhibitor of respiration, which are known to reduce mitochondrial superoxide generation in mammalian cells and yeast. These inhibitors increased viability at day nine and eleven by 2-3 fold (FIG. 2D) (p<0.05). Since respiration is essential for long-term survival, and FCCP and NaCN inhibit energy production by the mitochondria, the experiments could only be carried out to day eleven.

### Example 7

### Aconitase Activity and Reactivation

To study further the role of superoxide in the aging and death of S. cerevisiae we measured the activity of aconitase, a mitochondrial 4Fe-4S cluster-containing enzyme sensitive to inactivation by superoxide. Using cell extracts from two experiments, we measured aconitase activity in five independent wild type populations with mortality rates at day 5 ranging from 0.44 to 0.9 (High Mortality, HM), and five SOD1SOD2 overexpressors with mortality rates ranging from 0 to 0.37 (Low Mortality, LM) (FIG. 8A). Mortality rates at day n represent the percentage of the population that died between day n and day n+2. In both the HM and LM groups, aconitase activity was high at day 3 (FIG. 8B). At day 5 aconitase activity was 6 fold higher in the LM compared to the HM group (FIGS. 8A, B) suggesting that loss of aconitase activity precedes, and may contribute to, death in non-dividing yeast. The partial inactivation of aconitase in the LM group at day 5 is not surprising considering that mortality rates in this group are low at day 5 but increase dramatically in the following four days.

The exposure of aconitase and of other 4Fe-4S clusters-containing enzymes to superoxide causes inactivation due to the oxidation-dependent loss of one iron from the 4Fe-4S cluster. Aconitase can be reactivated by incubation of cell extracts with excess Fe³⁺ and sulfide (S²⁻). Little reactivation occurred for either the HM and LM groups at day three (FIG. 8B). By contrast, at day five, incubation of extracts with Fe³⁺ and S²⁻ caused a 15-fold reactivation of aconitase in HM extracts and a 5-fold reactivation in LM extracts (FIG. 8B), suggesting that the enzyme was present but was inactive due to the loss of iron from its 4Fe-4S cluster. Reactivation of aconitase by more than 10-fold was also observed in HM and LM extracts on day 7 (data not shown).

To test the effect of aconitase inactivation and loss of mitochondrial function on survival we treated cells with agents known to inactivate aconitase in a superoxide-dependent manner (antimycin A, paraquat) and monitored the survival of a mutant that is respiration deficient (coq3Δ). Treatment of wild type cells with 1 μM antimycin A or 1 mM paraquat, which increases the generation of mitochondrial superoxide and reversibly inactivates aconitase, resulted in an early viability loss (FIG. 8C). These results are consistent with a role for mitochondrial superoxide in the inactivation of aconitase and early loss of viability. The requirement for functional mitochondria during survival was confirmed by deleting COQ3, an enzyme involved in the biosynthesis of coenzyme Q, which is required for the function of mitochondrial complex III. coq3Δ mutants died by day 4 (FIG. 8C).

### Example 8

### Survival of ras Mutants

In yeast, Ras1 and Ras2 activate adenylate cyclase (Cyr1). To identify proteins that regulate longevity upstream of Cyr1, we measured the life span of ras1 and ras2 deletion mutants. Deletion of RAS1 in strain SP1 slightly decreased survival (data not shown), but the deletion of RAS2 doubled survival (FIG. 9A, B) (p<0.05). ras2 null mutations increased the mean life span by over 100% in both wild type strains SP1 and DBY746 (FIGS. 9A, B) and decreased mortality rates between day five and nineteen by 4 to 60 fold in the SP1 background and by 1.5 to 5 fold in the DBY746 background (data not shown). To confirm the role of Ras2 in longevity we tested strains carrying temperature sensitive (ts) mutations in the Ras pathway. ras1-ras2^{ts} (lacking RAS1 and with a temperature sensitive mutation in RAS2) maintained at the restrictive but not at the permissive temperature doubled survival compared to wild type controls (data not shown). The constitutive activation of Ras2 in RAS2val19 mutants cells sharply decreased survival (FIG. 9C). These results confirm that the Ras2/cAMP/PKA pathway regulates the chronological life span. Bacterial populations can grow by using nutrients released by dead cells (gasping). Since yeast ras1-ras2^{ts} are unable to divide at the restrictive temperature, these results also strongly suggest that the increased viability of ras2 mutants at advanced ages is not the result of “gasping”.

### Example 9

### Ras2, Msn2/Msn4 and SOD2

To test whether ras2 mutants were resistant to oxidative stress analogously to cyr1::mtn and sch9 mutants we treated mutant strains with the superoxide-generating agent paraquat. ras2 mutants retained over 70% of the initial viability after a 7-day treatment with paraquat (1 mM) compared to the 5% survival for paraquat-treated wild type controls (FIG. 10A). To test the role of stress resistance genes in the extended longevity of ras2Δ mutants we deleted transcription factors Msn2/Msn4 in ras2Δ. The deletion of msn2Δmsn4Δ abolished the effect of ras2Δ on longevity confirming that Ras2 and Cyr1 function in the same pathway to down-regulate stress-resistance and promote senescence (FIG. 10B, p<0.05). To test whether superoxide dismutases function downstream of Msn2/Msn4 to regulate survival extension in ras2Δ mutants we deleted SOD2 in ras2Δ mutants (ras2Δsod2Δ, PF106). The survival of ras2Δ mutants was clearly shortened by the deletion of SOD2 (FIG. 10C) (p<0.05). However, ras2Δsod2Δ survived 30% longer than wild type cells (p<0.05) indicating that the induction of other systems is important for survival extension. To test whether increasing superoxide protection could extend further the survival of ras2Δ mutants we overexpressed both SOD1 and SOD2 in ras2Δ mutants. ras2Δ SOD1SOD2ox mutants survived slightly shorter than ras2Δ mutants indicating that ras2Δ cells have optimized their protection against superoxide toxicity (data not shown).

### Example 10

### Age-dependent Metabolic Rates

To characterize further the chronological life span and test whether survival extension is associated with an early decrease in metabolic rates we measured oxygen consumption in long-lived mutants. In two wild type strains (DBY746, SP1), respiration was low when the cells were actively growing in log phase, increased during the diauxic shift and remained high until day five or six (FIGS. 11A, B). In sch9A mutants, the age-dependent oxygen consumption was similar to that of wild type cells (FIG. 6A). Metabolic rates in the DBY746 background decreased 48 hours earlier in ras2Δ and cyr1::mTn mutants than in wild type cells. However, in the SP1 background the age-dependent oxygen consumption for ras2Δ was similar to that of wild type cells (FIG. 6B). Neither SOD1SOD2 nor SOD1CTT1 overexpression had significant effects on the age-specific metabolic rates compared to DBY746 plasmid controls (data not shown). These results suggest that an early decrease in metabolic rates is associated with certain mutations that extend survival but is not required for longevity extension.

### Example 11

### Age-dependent Mitochondrial Function

Macromolecular damage in the mitochondria may contribute to aging and aging-related diseases in mammals. In most organisms it is very difficult to assess whether mitochondrial damage is a primary cause of aging and cell death. A valuable feature of yeast cells is the ability to survive without functional mitochondria, which allows the detection of the loss of mitochondrial function when the cell is viable. For this purpose we measured the index of respiratory competence (IRC), defined as the portion of cells able to grow using a carbon source that requires functional mitochondria as a percentage of all the viable yeast that can grow by glucose fermentation. For this experiment, we chose a wild type isolate with particularly high mortality rates and we studied survival after switching cells to water on day 3. This switch extends longevity and allows the monitoring of the IRC for a longer period compared to incubation in minimal medium. In all the experiments performed, during the growth phase and during the first three days of survival, the IRC remained close to 100%. Starting at day five, during the high mortality phase, a 20-30% drop in the IRC was observed (FIG. 12), suggesting that this fraction of the viable population had lost the ability to utilize mitochondrial respiration for growth. Similar results were obtained with another non-fermentable carbon source (lactate) (data not shown). These results are consistent with the age-dependent reversible inactivation of mitochondrial aconitase and with the role of mitochondrial SOD2 in longevity extension.

### Example 12

### Survival in the Reproductive and Post-reproductive Phase

The chronological life span in the post-diauxic phase is measured by monitoring the ability of a cell to form a colony within 3 days of incubation on complete medium (Colony Forming Units or CFU). We tested whether the loss of CFU correlates with the death of the organism. We measured the concentration of proteins released into the medium by dead and damaged wild type DBY746-plasmid control cells and by the longer-lived SOD1SOD2 double overexpressors. The increase in protein concentration in the medium of both strains began within two days of the major loss of CFUs at day 10 (FIG. 13). The increase in protein concentration in the medium of SOD1SOD2 overexpressors, which survive 2 days longer, was delayed by two days compared to controls (FIG. 8A). The release of protein by SOD1SOD2 overexpressors was lower compared to wild type controls throughout the study. Taken together these results suggest that the loss of the ability to form a colony (CFU) is followed by death and lysis and is a valid method to estimate the total chronological life span of yeast, which includes the reproductive and post-reproductive phases (FIG. 13B).

All of the publications which are cited in the body of the instant specification or listed below are hereby incorporated by reference in their entirety.

It is also to be appreciated that the foregoing description of the invention has been presented for purposes of illustration and explanation and is not intended to limit the invention to the precise manner of practice herein. It is to be appreciated therefore, that changes may be made by those skilled in the art without departing from the spirit of the invention and that the scope of the invention should be interpreted with respect to the following claims.

TABLE I
Mitochondrial aconitase specific activity (percent of day 3) in whole
cell extracts of yeast strains removed from day 4-7 cultures.

| Day | wt | sch9::Tn3 | cyr1::Tn3 | sch9Δ |
|---|---|---|---|---|
| 4 | 68.7 | 94.1 | 81.4 | 94 |
| 5 | 75 | 113.1 | 74.2 | 106 |
| 6 | 53 | 131.2 | 62.8 | 104.8 |
| 7 | 51.8 | 116 | 69.6 | 93.8 |

TABLE II
Yeast strains used in Examples 5-11.

| | | |
|---|---|---|
| Strain | Geonotype | Source |

| | |
|---|---|
| DBY746 | MATα leu 2-3, 112 his3Δ1 trp1-289 ura 3-52 GAL⁺ |
| SP1 | MATα leu2 his3 ura3 trp1 ade8 can1 |
| KP-1b | SP1 ras2::URA3 |
| PF101 | DBY746 cyr1::mTn |
| EG252 | DBY746 ras2::LEU2 |
| PF102 | DBY746 sch9::URA3 |
| EG110 | DBY746 sod2::TRP1 |
| PF103 | DBY746 msn2::HIS3 msn4::LEU2 |
| PF104 | DBY746 ras2::LEU2 sod2::TRP1 |
| PF105 | DBY746 cyr1::mTn sod2::TRP1 |
| PF106 | DBY746 sch9::URA3 sod2::TRP1 |
| PF107 | DBY746 ras2::LEU2 msn2::HIS3 msn4::LEU2 |
| TK1611R2V | SP1 RAS2val19 |
| CC103 | DBY746 coq3::LEU2 |

TABLE III
Specific activities of Sod1, Sod2 and catalase (units/mg)

| Strain | CuZnSOD (Sod1) | MnSOD (Sod2) | Catalase (Ctt1) |
|---|---|---|---|
| 351-352 | 0.48 | 0 | 3.9 |
| SOD1-SOD2 | 1.97 | 0.42 | ND |
| SOD1-CTT1 | ND | ND | 11.6 |

### References and Notes

### References

1. T. Finkel, N. J. Holbrook, Nature 408, 239-47 (2000).

2. V. D. Longo, Neurobiol Aging 20,479-86 (1999).

3. C. E. Finch, Longevity, Senescence, and the Genome (University Press, Chicago, 1990).

4. J. W. Vaupel, et al., Science 280, 855-60 (1998).

5. N. K. Egilmez, S. M. Jazwinski, J Bacteriol 171, 37-42 (1989).

6. D. Sinclair, K. Mills, L. Guarente, Annu Rev Microbiol 52, 533-60 (1998).

7. S. J. Lin, P. A. Defossez, L. Guarente, Science 289, 2126-8. (2000).

8. V. D. Longo, L. M. Ellerby, D. E. Bredesen, J. S. Valentine, E. B. Gralla, J. Cell Biol. 137, 1581-8 (1997).

9. Lee-Loung Liou, Paola Fabrizio, Vanessa N. Moy, James W. Vaupel, , Joan SelverstoneValentine, Edith Butler Gralla, and Valter D. Longo (Unpublished results)(Lee Loung Liou, Ph.D. Thesis University of California Los Angeles, 1999).

10. M. Werner-Washburne, E. L. Braun, M. E. Crawford, V. M. Peck, Mol. Microbial 19, 1159-66 (1996).

11. V. D. Longo, E. B. Gralla, J. S. Valentine, J. Biol. Chem. 271, 12275-12280 (1996).

12. V. D. Longo, L. L. Liou, J. S. Valentine, E. B. Gralla, Arch. Biochem. Biophys. 365,131-142 (1999).

13. Transposon mutagenesis and allele rescue were performed with the yeast insertion library provided by M. Snyder as described by Ross-Macdonald, P et al, Methods Enzymol., 303, 512-532 (1999).

14. Supplementary material is available at Science Online at www.sciencemag.org/feature/data/1059497.shl

15. K. A. Morano, D. J. Thiele, Embo J 18, 5953-62 (1999).

16. J. M. Thevelein, J. H. de Winde, Mol Microbiol 33, 904-18 (1999).

17. L. Guarente, C. Kenyon, Nature 408, 255-62. (2000).

18. S. Paradis, M. Ailion, A. Toker, J. H. Thomas, G. Ruvkun, Genes Dev 13, 1438-52 (1999).

19. E. S. Kandel, N. Hay, Exp Cell Res 253, 210-29 (1999).

20. J. A. Flattery-O'Brien, C. M. Grant, I. W. Dawes, Mol. Microbiol. 23, 303-12 (1997).

21. Pedruzzi, N. Burckert, P. Egger, C. De Virgilio, Embo J 19, 2569-79 (2000).

22. P. R. Gardner, I. Fridovich, J Biol Chem 267, 8757-63 (1992).

23. T. E. Johnson, Science 249, 908-12 (1990).

24. S. Melov, et al., Science 289, 1567-9 (2000).

25. Y. J. Lin, L. Seroude, S. Benzer, Science 282, 943-6 (1998).

26. L. J. Yan, R. L. Levine, R. S. Sohal, Proc. Natl. Acad. Sci. USA 94, 11168-72 (1997).

27. T. L. Parkes, et al., Nat. Genet. 19,171-4 (1998).

28. J. Sun, J. Tower, Mol. Cell. Biol. 19, 216-28 (1999).

29. E. Migliaccio, et al., Nature 402, 309-13 (1999).

30. Smith, M. P. Ward, S. Garrett, EMBO J. 17, 3556-64 (1998).

31. M. T. Martinez-Pastor, S. C. Marchler G., A. Marchler-Bauer, H. Ruis, F. Estruc, Embo J. 15, 2227-2235 (1996).

32. A. P. Schmitt, McEntee K., PNAS 93, 5777-5782 (1996).

33. J. A. Flattery-O'Brien, C. M. Grant, I. W. Dawes, Mol. Microbiol. 23, 303-12 (1997).

34. S. Paradis, G. Ruvkun, Genes Dev 12, 2488-98 (1998).

35. T. E. Johnson, Science 249, 908-12 (1990).

36. J. Z. Morris, Tissebaum, H. A., and Ruvkun G., Nature 382, 536-539 (1996).

37. K. D. Kimura, H. A. Tissenbaum, Y. Liu, G. Ruvkun, Science 277, 942-6 (1997).

38. S. Ogg, et al., Nature 389, 994-9 (1997).

1a . Werner-Washburne, M., Braun, E., Johnston, G. C. & Singer, R. A. (1993) Microbiol. Rev. 57, 383-401.

2a. Zambrano, M. M. & Kolter, R. (1996) Cell 86, 181-4.

3a. Werner-Washburne, M., Braun, E. L., Crawford, M. E. & Peck, V. M. (1996) Mol Microbiol. 19, 1159-66.

4a. Lillie, S. H. & Pringle, J. R. (1980) J. Bacteriol. 143, 1384-1394.

5a. Granot, D. & Snyder, M. (1991) Proc. Natl. Acad. Sci. (USA) 88, 5724-5728.

6a. Longo, V. D., Ellerby, L. M., Bredesen, D. E., Valentine, J. S. & Gralla, E. B. (1997) J. Cell Biol. 137, 1581-8.

7a. Longo, V. D., Liou, L. L., Valentine, J. S. & Gralla, E. B. (1999) Arch. Biochem. Biophys. 365, 131-142.

8a. Sinclair, D., Mills, K. & Guarente, L. (1998) Annu. Rev. Microbiol. 52, 533-60.

9a. Jazwinski, S. M. (1996) Science 273, 54-9.

10a. Ashrafi, K., Sinclair, D., Gordon, J. I. & Guarente, L. (1999) Proc. Natl. Acad. Sci. USA 96, 9100-9105.

11 a. Kennedy, B. K., Austriaco, N. R. J., Zhang, J. & Guarente, L. (1995) Cell 80, 485-496.

12a. Sun, J., Kale, S. P., Childress, A. M., Pinswasdi, C. & Jazwinski, S. M. (1994) J. Biol. Chem. 269, 18638-45.

13a. Sinclair, D. A., Mills, K. & Guarente, L. (1997) Science 277, 1313-6.

14a. Kane, D. J., Sarafian, T. A., Anton, R., Hahn, H., Gralla, E. B., Valentine, J. S., Ord, T. & Bredesen, D. E. (1993) Science 262, 1274-7.

15a. Longo, V. D., Gralla, E. B. & Valentine, J. S. (1996) J. Biol Chem. 271, 12275-12280.

16a. Toda, T., Uno, I., Ishikawa, T., Powers, S., Kataoka, T., Broek, D., Cameron, S., Broach, J., Matsumoto, K. & Wigler, M. (1985) Cell 40, 27-36.

17a. Roberts, R. L., Mosch, H. U. & Fink, G. R. (1997) Cell 89, 1055-65.

18a. Smith, A., Ward, M. P. & Garrett, S.(1998) EMBO J. 17, 3556-64.

19a. Martinez-Pastor, M. T., Marchler G., S. C., Marchler-Bauer, A., Ruis, H. & Estruc, F. (1996) Embo J. 15, 2227-2235.

20a. Ruis, H. & Schuller, C. (1995) Bioessays 17, 959-65.

21a. Flattery-O'Brien, J. A., Grant, C. M. & Dawes, I. W. (1997) Mol. Microbiol. 23, 303-12.

22a. Belazzi, T., Wagner, A., Wieser, R., Schanz, M., Adam, G., Hartig, A. & Ruis, H. (1991) Embo J. 10, 585-592.

23a. Mitsuzawa, H., Uno, I., Oshima, T. & Ishikawa, T. (1989) Genetics 123, 739-48.

24a. Racker, E. (1950) Biochim. Biophys. Acta 4, 211-214.

25a. Turrens, J. F., Alexandre, A. & Lehninger, A. L. (1985) Arch. Biochem. Biophys. 237, 408-14.

26a. Boveris, A. a. C., B. (1973) Biochem. J. 134, 707-716.

27a. Melov, S., Coskun, P. E. & Wallace, D.C. (1999) Mutat Res 434, 233-42.

28a. Beckman, K. B. & Ames, B. N. (1998) Physiological Reviews 78, 547-81.

29a. Li, Y., Huang, T. T., Carlson, E. J., Melov, S., Ursell, P. C., Olson, J. L., Noble, L. J., Yoshimura, M. P., Berger, C., Chan, P. H. & et al. (1995) Nat. Genet. 11, 376-81.

30a. Fridovich, I. (1995) Annu. Rev. Biochem. 64, 97-112.

31a. Flint, D. H., Tuminello, J. F. & Emptage, M. H. (1993) Journal of Biological Chemistry 268, 22369-76.

32a. Jonassen, T. & Clarke, C. F. (2000) J Biol Chem 275, 12381-7.

33a. Gardner, P. R., Raineri, I., Epstein, L. B., and White, C. W. (1995) J. Biol. Chem. 270, 13399-13405.

34a. Pichova, A., Vondrakova, D. & Breitenbach, M. (1997) Can J Microbiol 43, 774-81.

35a. Kirchman, P. A., Kim, S., Lai, C. Y. & Jazwinski, S. M. (1999) Genetics 152, 179-90.

36a. Muller, I., Zimmermann, M., Becker, D. & Flomer, M. (1980) Mech. Ageing Dev. 12, 47-52.

37a. Kenyon, C., Chang, J., Gensch, E., Rudner, A. & Tabtiang, R. (1993) Nature 366, 461-4.

38a. Johnson, T. E. (1990) Science 249, 908-12.

39a. Kimura, K. D., Tissenbaum, H. A., Liu, Y. & Ruvkun, G. (1997) Science 277, 942-6.

40a. Larsen, P. L. (1993) Proc. Natl Acad. Sci. USA 90, 8905-8909.

41a. Lithgow, G. J., White, T. M., Melov, S. & Johnson, T. E. (1995) Proc. Natl. Acad. Sci. USA 92, 7540-4.

42a. Orr, W. C. & Sohal, R. S. (1994) Science 263, 1128-30.

43a. Sun, J. & Tower, J. (1999) Mol. Cell. Biol. 19, 216-28.

44a. Parkes, T. L., Elia, A. J., Dickinson, D., Hilliker, A. J., Phillips, J. P. & Boulianne, G. L. (1998) Nat. Genet. 19, 171-4.

45a. Lin, Y. J., Seroude, L. & Benzer, S. (1998) Science 282, 943-6.

46a. Yan, L. J., Levine, R. L. & Sohal, R. S. (1997) Proc. Natl. Acad. Sci. USA 94, 11168-72.

47a. Longo, V. D. (1999) Neurobiol Aging 20, 479-86.

48a. Migliaccio, E., Giorgio, M., Mele, S., Pelicci, G., Reboldi, P., Pandolfi, P. P., Lanfrancone, L. & Pelicci, P. G. (1999) Nature 402, 309-13.

49a. Finkbeiner, S. & Greenberg, M. E. (1996) Neuron 16, 233-6.

50a. Ferrari, G. & Greene, L. A. (1994) EMBO J. 13, 5922-8.

51a. Spear, N., Estevez, A. G., Johnson, G. V., Bredesen, D. E., Thompson, J. A. & Beckman, J. S. (1998) Arch. Biochem. Biophys. 356, 41-5.

52a. Mills, E. M., Takeda, K., Yu, Z. X., Ferrans, V., Katagiri, Y., Jiang, H., Lavigne, M. C., Leto, T. L. & Guroff, G. (1998) J. Biol. Chem. 273, 22165-8.

53a. Henkemeyer, M., Rossi, D. J., Holmyard, D. P., Puri, M. C., Mbamalu, G., Harpal, K., Shih, T. S., Jacks, T. & Pawson, T. (1995) Nature 377, 695-701.

54a. Xia, Z., Dickens, M., Raingeaud, J., Davis, R. J. & Greenberg, M. E. (1995) Science 270, 1326-31.

55a. Wilson, D. J., Fortner, K. A., Lynch, D. H., Mattingly, R. R., Macara, I. G., Posada, J. A. & Budd, R. C. (1996) Eur. J. Immunol. 26, 989-94. Marte, B. M. & Downward, J. (1 997) Trends in Biochemical Sciences 22, 3558.

What is claimed is:

1. An agent for extending the life-span of a eukaryote, wherein said agent modulates a pathway which involves the participation of a product of at least one gene selected from the group consisting of a ras gene, SOD2, Sch9, MSN2, MSN4, RIM15 and homologs thereof.

2. The agent of claim 1, wherein the eukaryote is a mammal.

3. The agent of claim 2, wherein the mammal is a human.

4. An agent for extending the life-span of a eukaryote, wherein said agent modulates a signal transduction pathway that regulates multiple stress resistance systems.

5. The agent of claim 4, wherein the eukaryote is a mammal.

6. The agent of claim 5, wherein the mammal is a human.

7. An agent for extending the life-span of a eukaryote, wherein said agent modulates a signal transduction pathway that regulates SOD2 activity.

8. The agent of claim 7, wherein the eukaryote is a mammal.

9. The agent of claim 8, wherein the mammal is a human.

10. An agent for extending the life-span of a eukaryote, wherein said agent regulates the expression of genes encoding for heat shock proteins, genes encoding for catalase, or the DDR2 gene.

11. The agent of claim 10, wherein the eukaryote is a mammal.

12. The agent of claim 11, wherein the mammal is a human.

13. An agent for extending the life-span of a eukaryote, wherein said agent modulates a pathway that depends on the activity of at least one polypeptide selected from the group consisting of Msn2, Msn4, Rim-15 and homologs thereof.

14. The agent of claim 13, wherein the eukaryote is a mammal.

15. The agent of claim 14, wherein the mammal is a human.

16. An agent for extending the life-span of a eukaryote, wherein said agent modulates a pathway that is activated in response to glucose or other nutrients.

17. The agent of claim 16, wherein the eukaryote is a mammal.

18. The agent of claim 17, wherein the mammal is a human.

19. A method for increasing the life-span of a eukaryote, the method comprising contacting the cell of the eukaryote with the agent of claim 1, claim 4, claim 7, claim 10, claim 13 or claim 16.

20. A system for studying the aging and death of a eukaryote, the system comprising a long-lived yeast mutant.

21. The system of claim 20, wherein the eukaryote is a mammal.

22. The system of claim 21, wherein the mammal is a human.

23. A method for extending the life-span of a eukaryote, the method comprising modulating a pathway which involves the participation of a product of at least one gene selected from the group consisting of a ras gene, SOD2, Sch9, MSN2, MSN4, RIM15 and homologs thereof.

24. The method of claim 23, wherein the eukaryote is a mammal.

25. The method of claim 24, wherein the mammal is a human.

26. A method for extending the life-span of a eukaryote, the method comprising modulating a signal transduction pathway that regulates multiple stress resistance systems.

27. The method of claim 26, wherein the eukaryote is a mammal.

28. The method of claim 27, wherein the mammal is a human.

29. A method for extending the life-span of a eukaryote, the method comprising modulating a signal transduction pathway that regulates SOD2 activity.

30. The method of claim 29, wherein the eukaryote is a mammal.

31. The method of claim 30, wherein the mammal is a human.

32. A method for extending the life-span of a eukaryote, the method comprising regulating the expression of genes encoding for heat shock proteins, genes encoding for catalase, or the DDR2 gene.

33. The method of claim 32, wherein the eukaryote is a mammal.

34. The method of claim 33, wherein the mammal is a human.

35. A method for extending the life-span of a eukaryote, the method comprising modulating a pathway that depends on the activity of at least one polypeptide selected from the group consisting of Msn2, Msn4, Rim-15 and homologs thereof.

36. The method of claim 35, wherein the eukaryote is a mammal.

37. The method of claim 36, wherein the mammal is a human.

38. A method for extending the life-span of a eukaryote, the method comprising modulating a pathway that is activated in response to a nutrient.

39. The method of claim 38, wherein the nutrient is glucose.

40. The method of claim 39, wherein the eukaryote is a mammal.

41. The method of claim 40, wherein the mammal is a human.
